(19) United States
(12) Patent Application Publication
(10) Pub. No.: US 2020/0385789 A1
(43) Pub. Date: Dec. 10, 2020

(54) METHODS OF ASSESSING TELOMERES

(71) Applicant: Children's Medical Research Institute, Westmead (AU)

(72) Inventors: Hilda PICKETT, Surry Hills (AU); Joshua ALLEN, Petersham (AU)

(73) Assignee: Children's Medical Research Institute, Westmead (AU)

(21) Appl. No.: 16/643,398

(22) PCT Filed: Aug. 31, 2018
(86) PCT No.: PCT/AU2018/050942

§ 371 Date: Feb. 28, 2020

(30) Foreign Application Priority Data
Sep. 1, 2017 (AU) 2017903528

(51) Int. Cl. C12Q 1/6813; C12Q 1/6806; G16B 25/00; G06K 9/00; G06K 9/46; G06T 7/13; G06T 7/187; G06K 9/62; G06T 5/00; G06T 7/60

(52) U.S. Cl. CPC C12Q 1/6813; G06T 2207/30004; G16B 25/00; G06K 9/0014; G06K 9/4652; G06K 9/4638; G06T 7/13; G06T 7/187; G06K 9/00147; G06K 9/6228; G06T 5/001; G06T 5/007; G06K 9/6288; G06T 7/60; G06K 9/00134; G06T 2207/20212; G06T 2207/10056; C12Q 1/6806

(57) ABSTRACT

This invention relates generally to methods for assessing telomeres, including methods of measuring the length of a telomere and methods for detecting extension of a telomere and methods for measuring telomere extension, and associated systems.

### FIELD OF THE INVENTION

This invention relates generally to methods for assessing telomeres, including methods of measuring the length of a telomere and methods for detecting extension of a telomere and methods for measuring telomere extension, and associated systems.

### RELATED APPLICATIONS

This application claims priority to Australian Provisional Application No. 2017903528 entitled “Methods of assessing telomeres” filed 1 Sep. 2017, the contents of which are incorporated herein by reference in their entirety.

### BACKGROUND OF THE INVENTION

Telomeres are structures present at the ends of the chromosomes of eukaryotic organisms which contain repetitive nucleic acid sequences (telomere repeats or telomere repeat units) and are produced by the telomerase enzyme complex, which itself contains two molecules each of the telomerase reverse transcriptase (TERT), telomerase RNA (TR or TERC) and dyskerin (DKC1), and functions to add new telomeric repeat units to the chromosome termini. The telomere repeat units have a sequence that is compatible with the telomerase enzyme particular to the organism. In vertebrates, including humans, this repeat unit is TTAGGG (CCCTAA). In addition to the double-stranded repeat sequences, the 3′ end contains a single-stranded overhang, which folds back on itself to form a large loop structure called a telomere loop or T-loop. In human somatic cells, each telomere is typically between 4-12 kb long and the single-stranded overhang generally contains 100-200 nucleotides. The telomeric DNA is protected from normal DNA repair mechanisms, at least in part, by the telomere-binding proteins, which include telomeric repeat binding factors 1 and 2 (TRF1 and TRF2), protection of telomeres 1 (POT1), TRF1 and TRF2 interacting nuclear protein 2 (TIN2), the human ortholog of the yeast repressor/activator protein 1 (Rap1), and TPP1. Together these proteins form a complex known as shelterin, which binds to the telomeric DNA.

Telomeres are vital structures, protecting chromosome ends from DNA degradation, recombination and DNA end fusions, as well as being important for nuclear architecture. During DNA replication in eukaryotes, the full length of the chromosome is not copied completely, resulting in progressive loss of 15-66 bp per year, with the fastest rate in the first year of life, slowing to then again increase over the age of 50 (see e.g. Barrett et al. Hum Genet. 2015 134(7): 679-689). The presence of telomeres at the end of the chromosome means that this loss of nucleic acid occurs in the non-coding telomeric region rather than the coding regions of the chromosome, thus maintaining the integrity of the coding regions. However, the gradual shortening of the telomeres over the various cycles of cell division ultimately results in cell cycle arrest leading to apoptosis or senescence. Thus, telomere length is often considered a biomarker of chronological aging.

Although telomerase is constitutively active in germline cells and in some hematopoietic cells, it is tightly repressed in normal somatic cells. This lack of telomerase activity contributes to the loss of telomere length described above, and eventual cell senescence or death. However, many cancer cells are able to overcome this cell senescence or death by upregulating the activity of telomerase in a cell so that new telomere repeats are added to the termini, and/or by utilizing a telomerase-independent mechanism, termed Alternative Lengthening of Telomeres (ALT), which involves a homologous recombination-based DNA replication mechanism to extend telomere length (for review, see e.g. Jafri et al. Genome Medicine, 2016, 8:69; Pickett and Reddel, Nat Struct Mol Biol. 2015, 22(11):875-80; and Dilley and Greenberg, Trends Cancer. 2015 Oct. 1; 1(2): 145-156).

Telomere length has been associated with a variety of diseases and conditions, and has been used as a diagnostic and prognostic marker of these conditions. Severe telomere length deficiencies (typically falling in the bottom percentile of the population) give rise to short telomere syndromes or telomeropathies. These are rare but serious syndromes that arise from the loss of critical stem cell populations, and manifest in pulmonary fibrosis, bone marrow failure, aplastic anemia and myelodysplastic syndrome/acute myeloid leukemia. Short telomere length has also been associated with cardiovascular disease, chronic obstructive pulmonary disease and diabetes. Telomere length has also been associated with cancer risk and prognosis, although this link is far more complex and seemingly dependent on the type of cancer, where both long and short telomeres have been associated with an increased risk of developing a variety of cancers (see e.g. Zhu et al. Scientific Reports 2016 6:22243; Haycock et al. JAMA Oncol. 2017 3(5):636-651). Adding a further layer of complexity is the fact that cancer cells override the natural replicative senescence and activate telomere lengthening mechanisms, thereby maintaining or lengthening telomeres and achieving proliferative immortality. Because of the association between telomere length and disease, there has been an increased focus on the development of agents that regulate telomere length (i.e. either promote or inhibit telomere extension) as potential therapeutics for these diseases (see e.g. Jager and Walter. Genes (Basel). 2016 7(7): 39).

Multiple methods to measure telomere length (e.g. for diagnostic and prognostic use) have been developed over the years, although each has its limitations and there is currently no single technique to accurately, easily and rapidly measure the length of telomeres (for review, see e.g. Aubert et al. Mutat Res. 2012, 730(1-2): 59-67. Montpetit et al. Nurs Res. 2014, 63(4): 289-299).

One of the first methods developed to measure telomere length was terminal restriction fragment length analysis (TRF), which measures the average length of fragments resulting from digestion of genomic DNA with restriction enzymes that do not cleave telomeric and subtelomeric sequences, but that do cleave frequently within other genomic sequences. The resulting terminal restriction fragment, which contains both telomere repeats and subtelomeric DNA, is visualized by electrophoresis and hybridization with a telomere-specific probe. TRF length analysis is limited in its application by requiring large quantities of DNA. Moreover, it is only able to provide a range or average of lengths (i.e. can not be used to measure individual telomere lengths) and generally overestimates the actual average because it necessarily includes some subtelomeric region in the fragment. The inability of the TRF technique to clearly identify and quantitate very short telomeres is problematic as it is the shortest telomere(s), not average telomere length, that triggers cellular senescence (Hemann et al. Cell. 2001; 107:67-77; Abdallah et al. Nature Cell Biology. 2009; 11:988-993).

Flow-FISH is a technique commonly used in clinical testing. In this technique, cells are hybridized with fluorescently-labeled telomere-specific peptide nucleic acid (PNA) probes and then analyzed by flow cytometry. An advantage of Flow-FISH is that it can measure the telomere length of specific cell populations (e.g. using antibodies specific for various cell types). However, it is labor intensive and limited to fresh blood samples. Moreover, it provides only a relative measure of average telomere content in cells rather than providing absolute telomere length measurements. As noted above, given that it is the shortest telomeres and not average telomere length that triggers cellular senescence, Flow-FISH therefore lacks the ability to provide some critical, clinically-important information.

Quantitative PCR (qPCR) is used extensively in laboratory and epidemiological studies to measure telomere length due to the requirement for only small amounts of DNA, the relative accessibility of PCR and its compatibility with high throughput platforms. However, qPCR is widely considered to be highly error-prone and variable. In addition, like Flow-FISH, it provides only relative measurements of average telomere lengths in a cell.

Single Telomere Length Analysis (STELA) was developed as a method to measure individual telomere lengths rather than the average length. Despite the advantage that STELA provides in measuring individual telomere lengths from very small amounts of DNA, it still has significant limitations. Due to the requirement for primers that specifically bind the subtelomeric regions, STELA can only be used to measure telomere lengths on the small subset of chromosomes for which appropriate primers can be designed (e.g. XpYp, 2p, 11q, 12q, and 17p). Moreover, STELA is technically challenging to perform, is labor intensive and is not conducive to high throughput analysis, thereby limiting its use in large-scale clinical applications.

Various methods have also been developed for detecting and/or measuring telomere extension. Such methods are of increasing importance and utilization given the increased focus on developing and identifying potential therapeutic agents that regulate telomere length (i.e. either promote or inhibit telomere extension). However, many of these methods assess telomerase activity (for review, see e.g. Zhou and Wang et al. Chem. Sci., 2017, 8, 2495-2502) and are not effective in detecting telomere extension resulting from usage of the ALT pathway. For example, in the commonly-used PCR-based telomeric repeat amplification protocols (TRAPs), telomerase is extracted from cells and used to extend synthetic substrates before the extension products are amplified by PCR and detected.

There remains a need for alternative methods to assess telomeres. In particular, there remains a need for methods that are conducive to high-throughput analysis and that provide an absolute measurement of individual telomere length so that the information is more clinically-relevant. There also remains a need for easy, accurate and efficient methods to detect and/or measure telomere extension events. Systems for use with these methods, including automated or at least partially-automated systems amenable to high-throughput applications, are also desirable.

### SUMMARY OF THE INVENTION

The present invention provides methods and systems for analyzing telomeres, including determining the length of a telomere, detecting a telomere extension event and/or determining the length of a telomere extension or an extended telomere.

In one aspect, provided is a method for measuring the length of a telomere, comprising: stretching genomic DNA on to a surface of a support at a uniform stretching rate; hybridizing a telomere-specific probe to the DNA to obtain a probe-DNA hybrid; and detecting the probe-DNA hybrid to thereby determine the length of a telomere. In particular embodiments, the length of the telomere is determined by visualizing and measuring the absolute length of the probe-DNA hybrid. In other embodiments, the length of the telomere is determined by measuring the intensity of a detectable signal on the telomere-specific probe within the probe-DNA hybrid.

A further aspect of the invention provides a method for detecting extension of a telomere, comprising: contacting a cell with a nucleotide analogue under conditions sufficient for DNA synthesis and incorporation of the nucleotide analogue into the genomic DNA of the cell; isolating the genomic DNA from the cell; stretching the genomic DNA on to a surface of a support at a uniform stretching rate; hybridizing a telomere-specific probe to the DNA to obtain a probe-DNA hybrid; and detecting the probe-DNA hybrid and the nucleotide analogue to thereby determine whether telomere extension has occurred, wherein the presence of a probe-DNA hybrid comprising the nucleotide analogue is indicative of the extension of a telomere. In some embodiments, the method further comprises measuring the length of the extension, the length of the telomere with the extension and/or the length of the telomere excluding the extension. Determining the length of the extension, the length of the telomere with the extension and/or the length of the telomere excluding the extension may be performed by, for example, visualizing and measuring the absolute length of the probe-DNA hybrid or by measuring the intensity of a detectable signal on the telomere-specific probe within the probe-DNA hybrid. In additional embodiments, the method further comprises contacting the cell with an agent that inhibits or promotes telomere extension.

In one embodiment, the uniform stretching rate is between 0.1 kb/μm and 10 kb/μm, between 0.5 kb/μm and 5 kb/μm, or between 1 kb/μm and 3 kb/μm, such as, for example, between 1.5 kb/μm and 2.5 kb/μm, e.g. 1.6, 1.7, 1.8, 1.9, 2.0, 2.1, 2.2, 2.3 or 2.4 kb/μm.

In some examples, the genomic DNA is stretched on to the surface by molecular combing, such as with a molecular combing machine.

In some embodiments, the telomere-specific probe hybridizes to one or more telomere repeat units. When the genomic DNA is human genomic DNA, the telomere repeat unit may comprises the sequence TTAGGG. Thus, in one example, the telomere-specific probe is a nucleic acid probe comprising the sequence CCCTAA or TTAGGG.

In particular embodiments, the telomere-specific probe is a peptide nucleic acid (PNA) probe. The telomere-specific probe may comprise, for example, a visually-detectable label, such as a fluorescent label.

In the methods of the invention that are directed to detecting extension of a telomere, the nucleotide analogue may comprises a visually-detectable label. In one example, the visually-detectable label is applied to the analogue after incorporation of the analogue into the DNA. In another example, the nucleotide analogue comprises the visually-detectable label prior to incorporation of the analogue into the DNA. The visually-detectable label may be, for example, a fluorescent label.

In one embodiment, the nucleotide analogue is a thymidine analogue selected from among chlorodeoxyuridine (CldU), bromoeoxyuridine (BrdU), iododeoxyuridine (IdU) and ethynyldeoxyuridine (EdU), or a fluorescent analogue selected from 2-aminopurine (2AP), pyrrolo-C (PyC), 1,3-diaza-2-oxophenothiazine (tC), 1,3-diaza-2-oxophenoxazine (tCO) and 7-nitro-1,3-diaza-2-oxophenothiazine (tCnitro).

The methods of the present invention may also comprise staining the genomic DNA, such as with a fluorescent dye (e.g. YOYO-1, TOTO-1, POPO-1, BOBO-1 or JOJO-1).

In some embodiments, the genomic DNA is obtained from frozen, fresh, or fixed cells or tissue. In a particular embodiment, the method first comprises a step of extracting genomic DNA from cells or tissue, such as frozen, fixed or fresh cells or tissue.

In one example, the support is a glass support, such as a silanized glass support (e.g. a glass support coated with vinyl silane). The glass support may be, for example, a microscope slide or coverslip.

In some embodiments, at least a portion of the method is automated.

In further aspects, provided is a method for measuring the length of a telomere, the method including, in one or more processing devices: receiving image data captured by an imaging device, the image data being indicative of one or more images of at least part of a surface of a support, the surface having stretched genomic DNA hybridized to a telomere-specific probe attached thereto; detecting a probe-DNA hybrid in the one or more images; measuring a length of the probe-DNA hybrid in the one or more images; and using the measured length to determine a telomere length indicator indicative of the telomere length.

In some embodiments, the method includes detecting a probe-DNA hybrid in accordance with pixel parameters of image pixels of the image, the pixel parameters including at least one of: a pixel colour; a pixel intensity; a pixel brightness; a pixel hue; and, a pixel saturation. The method may also include determining the length of the probe-DNA hybrid by at least one of: region growing; and, edge detection.

In one embodiment, the method includes detecting candidate features in the image using the pixel parameters; and detecting a probe-DNA hybrid at least partially in accordance with the candidate features. The method may also include determining an extent of a candidate feature; and detecting a probe-DNA hybrid in accordance with the determined extent. The method may additionally include comparing the extent to one or more thresholds; and detecting a probe-DNA hybrid in accordance with results of the comparison. In some embodiments, the method includes determining the extent by at least one of: region growing; and, edge detection. In a particular embodiment, the method includes selectively excluding candidate features in accordance with at least one of candidate feature size, candidate feature location, and candidate feature orientation; and detecting a probe-DNA hybrid from the remaining candidate features.

In particular embodiments, the method includes: performing enhancement to generate an enhanced image using at least one of contrast enhancement, hue enhancement, intensity enhancement, brightness enhancement, colour enhancement, and saturation enhancement; and detecting a probe-DNA hybrid using the enhanced image.

In further embodiments, the image data includes a plurality of images and wherein the method includes forming a composite image from the plurality of images; and detecting a probe-DNA hybrid using the composite image.

In still further embodiments, the method includes measuring a length of plurality of probe-DNA hybrids; and, statistically analyzing the measured lengths to determine at least one of a telomere length; and a telomere length distribution.

Also provided is a system for measuring the length of a telomere, the system including, one or more processing devices that: receive image data from an imaging device, the image data being indicative of one or more images of at least part of a surface of a support, the surface having stretched genomic DNA hybridized to a telomere-specific probe attached thereto; detect a probe-DNA hybrid in the one or more images; measure a length of the probe-DNA hybrid in the one or more images; and, use the measured length to determine a telomere length indicator indicative of the telomere length. In some embodiments, the one or more processing devices generate an indicator indicative of at least one of a telomere length and a telomere length distribution; and at least one of display and store the indicator. The one or more processing devices may control the imaging device to capture the one or more images. In some embodiments, the support is mounted on a stage, and the one or more processing devices control actuators to thereby relatively move the stage and imaging device to thereby capture the one or more images.

In one embodiment, the system includes a molecular combing machine that stretches the genomic DNA on to the surface; an applicator that applies the telomere-specific probe to the surface; and at least one transport mechanism for transporting the substrate from the molecular combing machine to the applicator and from the applicator to the stage. In a particular embodiments, the one or more processing devices control at least one of: the molecular combing machine; the applicator; and the at least one transport mechanism.

In still further aspects, a computer program product is provided, including computer executable code, which when executed by a suitably programmed processing system causes the processing system to: receive image data from an imaging device, the image data being indicative of one or more images of at least part of a surface of a support, the surface having stretched genomic DNA hybridized to a telomere-specific probe attached thereto; detect a probe-DNA hybrid in the one or more images; measure a length of the probe-DNA hybrid in the one or more images; and, use the measured length to determine a telomere length indicator indicative of the telomere length.

### BRIEF DESCRIPTION OF THE DRAWINGS

FIG. 1 provides images of DNA fibres that were stretched on to a coverslip at a constant stretching rate of 2 kb/μm using a molecular combing machine, stained with YOYO-1 and then viewed under a fluorescent microscope. DNA combing and staining that resulted in the desired straight fibres of a good density (A and B) are shown. DNA combing and staining that resulted in undesirable outcomes, including fibres not stuck to the coverslip properly (C), low density fibres (D), wavy fibres (E and F), and DNA shearing (G and H) are also shown.

FIG. 2 shows images of 10 kb control DNA fragments that have been stretched on to a coverslip at a constant stretching rate of 2 kb/μm using a molecular combing machine (A) or that have been manually stretched on to a microscope slide at a stretching rate of approximately 3.27 kb/μm (B), stained with YOYO-1 and then viewed under a fluorescent microscope.

FIG. 3 shows the length of individual DNA fragments in μm (A) and kb (B), calculated by either stretching 10 kb fragments on to a coverslip at a constant stretching rate of 2 kb/μm using a molecular combing machine (molecular combing method) or manually stretching 10 kb fragments on to a microscope slide at a stretching rate of approximately 3.27 kb/μm (manual DNA extension method) before staining the DNA fibres with YOYO-1 and viewing them under a fluorescent microscope.

FIG. 4 represents the results of a side-by-side comparison of the use of telomere fibre-FISH (TFF) or an alternative fibre-FISH method to measure the length of telomeres in U-2 OS cells. TFF (or “molecular combing method”) was performed by stretching DNA from U-2 OS cells at a constant stretching rate of 2 kb/μm using a molecular combing machine before the DNA fibres were hybridized with a TAMRA-labelled telomere-specific probe and viewed under fluorescent microscope (A). The alternative fibre-FISH method (or “manual DNA fibre method”) was performed by manually stretching the DNA from U-2 OS cells on to a microscope slide at a stretching rate of approximately 3.27 kb/μm before the DNA fibres were hybridized with a TAMRA-labelled telomere-specific probe and viewed under fluorescent microscope (B). Telomere length was calculated in μm (C) and kb (D).

FIG. 5 represents the results of a side-by-side comparison of the use of TFF or terminal restriction fragment (TRF) length analysis to measure the length of telomeres in a range of cell lines that utilize the alternative lengthening of telomeres (ALT) pathway (VA13, U20S, JFCF-6/T.1R, JFCF-6/T.1M, IIICF/c, GM847 and AG11395 cells) and cell lines in which telomerase is activated (JFCF-6/T.1F, GM639, HeLa, HT1080 wt, HTR and TCA cells). The use of TFF facilitated the measurement of both individual telomere lengths (A) and average telomere length (B) while TRF (C) only provides information on the average telomere length.

FIG. 6 represents the results of a side-by-side comparison of the use of TFF or TRF length analysis to measure the length of telomeres in HT1080 wild-type cells, increasing population doublings of HT1080 hTR cells (early, mid, late), and a control ALT cell line (VA13 or IIICF/c cells). The use of TFF facilitated the measurement of the average telomere length with standard deviation (A) and individual telomere lengths (B) while TRF (C) only provides information on the average telomere length.

FIG. 7 represents the results of a side-by-side comparison of the use of TFF, flow-FISH or qPCR to assess the length of telomeres in HeLa, HT1080 wild-type, U2-OS and IIICF/c cells. (A) TTF, with absolute length of individual telomeres presented (mean and 2×standard deviation also shown); (B) flow-FISH, with relative, average telomere length in each cell sample shown as molecules of equivalent soluble fluorochrome (MESH) (mean and 2×standard deviation also shown); (C) qPCR, with relative average telomere length expressed as relative T/S ratio (mean and 2×standard deviation also shown).

FIG. 8 shows the results of an analysis of telomere length following exposure of cells to aphidicolin and/or INK128 (an mTOR inhibitor—mTORi). IMR90 primary human foetal lung fibroblasts were plated and 1 day later were exposed to aphidicolin, mTORi, aphidicolin+mTORi, or DMSO alone (untreated). Cells were harvested a further 7 days later and telomere length was assessed by TFF. (A) Representative imaging data of DNA fibres: Left panel—expanded view of telomere fibre fish imaging data. Center and right panels—close up of the boxes 1-5 shown on the left panel. Boxes are drawn around the telomere signals and the length in μm and kb is indicated. (B). Telomere length presented in a Tukey box plot. “+” is the mean, **=P<0.01.

FIG. 9 shows a schematic of DNA fibres following a telomere extension assay in which cells were labelled with CldU and incubated to allow DNA synthesis, before genomic DNA was isolated and stretched on to a coverslip at a constant stretching rate (e.g. 2 kb/μm) using a molecular combing machine. The DNA fibres are hybridized with a labelled telomere-specific probe (black) and a labelled secondary antibody against an anti-CldU antibody (grey) before being viewed under a fluorescent microscope. The upper image shows a non-replicating telomere fibre with no CldU incorporation (solid black line), the middle image shows a replicating telomere fibre with CldU incorporation at the telomere and in an adjacent region (dashed grey and black adjacent solid grey), and the bottom image shows telomere synthesis (extension) characterized by a telomeric fibre with CldU incorporation strictly at one end of the telomere with no overlap into the adjacent regions (dashed grey and black adjacent solid black).

FIG. 10 is a graphical representation of a study quantifying telomere extension events in U-2 OS and IIICF/c cells stably expressing SLX4 and BLM. The mean number of extension events in >350 fibers±SEM was determined from 3 experiments (A). The extension event length was quantified by measuring the length of the telomere fiber with CldU incorporation (B). The length of the telomere without the extension (pre-extension; no CldU incorporation) was also quantified (C).

FIG. 11 is a flow chart of a further example of a method for measuring the length of a telomere.

FIG. 12 is a schematic diagram of an example of a system for measuring the length of the telomere.

FIGS. 13A to 13C are a flow chart of a specific example of method for measuring the length of the telomere using the system of FIG. 12.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “a telomere” means one telomere or more than one telomere.

As used herein, “and/or” refers to and encompasses any and all possible combinations of one or more of the associated listed items, as well as the lack of combinations when interpreted in the alternative (or).

The term “about”, as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

The term “contacting” or “exposing” as used herein refers to bringing a disclosed compound or agent and a cell or DNA together in such a manner that the compound or agent can interact with a target in the cell (e.g. DNA in the cell) or the DNA, respectively. Contacting can take place in vitro, ex vivo, or in vivo. In specific embodiments, the term “contacting” includes allowing a nucleoside analogue to enter a cell and be incorporated into the DNA of the cell.

The terms “complementary” and “complementarity” refer to polynucleotides (i.e., a sequence of nucleotides) related by the base-pairing rules. For example, the sequence “5′ A-G-T 3′,” is complementary to the sequence “3′ T-C-A 5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements.

The terms “disorder” and “disease” are used interchangeably herein to refer to an abnormal condition that impairs bodily functions, associated with specific symptoms and signs.

As used herein, the term “hybridization”, “hybridizes to” or “hybridizing” is used interchangeably with “binding” or “binds to”. Hybridization denotes the pairing of complementary nucleotide sequences to produce a nucleic acid hybrid, such as a DNA-DNA hybrid or PNA-DNA. Complementary base sequences are those sequences that are related by the base-pairing rules. In DNA and PNA, A pairs with T and C pairs with G. In RNA, U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently. For the purposes of the present invention, “hybridization”, “hybridizes to” or “hybridizing” is intended to describe conditions for moderate stringency or high stringency hybridization, preferably where the hybridization and washing conditions permit nucleotide sequences at least 60% homologous to each other to remain hybridized to each other. Preferably, the conditions are such that sequences at least about 70%, more preferably at least about 80%, even more preferably at least about 85%, 90%, 95% or 98% homologous to each other typically remain hybridized to each other. Stringent conditions are known to those skilled in the art and can be found in Current Protocols in Molecular Biology, John Wiley & Sons, N.Y. (1989), 6.3.1-6.3.6.

The term “oligomer” refers to a plurality of naturally-occurring and/or non-naturally-occurring nucleosides, joined together in a specific sequence, to form a polymeric structure. Included in the term “oligomer” are oligonucleotides, oligonucleotide analogs, oligonucleotide mimetics, oligonucleosides and chimeric combinations of these, and is thus intended to be broader than the term “oligonucleotide”, including all oligomers having all manner of modifications including but not limited to those known in the art. Oligomers are typically structurally distinguishable from, yet functionally interchangeable with, naturally-occurring or synthetic wild-type oligonucleotides. Thus, oligomers include all such structures that function effectively to mimic the structure and/or function of a desired DNA strand, for example, by hybridizing to a target. Such non-naturally occurring oligonucleotides are often desired over the naturally-occurring forms because they often have enhanced properties, such as for example, enhanced affinity for nucleic acid target and increased stability.

As defined herein, a “probe” is a polynucleotide, a polynucleotide/polypeptide hybrid or a polypeptide, which has the capacity to hybridize to target nucleic acid, in particular to DNA for the purposes of the present invention, by base pairing with the target nucleic acid. A probe is substantially or fully complementary to the target DNA and accordingly enables stable probe-DNA hybrids to be formed under stringent conditions of hybridization. Probes may comprise DNA, RNA, analogues such as peptide nucleic acid (PNA), locked nucleic acid (LNA), unlocked nucleic acid, (UNA) and triazole-linked DNA, and/or protein domains. A probe specific for a given target is a probe capable, under certain conditions, such as medium or high stringency, of hybridizing to a target in the DNA while, in the same conditions, not hybridizing to most or all other non-target sequences or regions in the DNA. In particular embodiments, a probe is an oligomer, having at least or about 8, 9, 10, 11, 12, 13, 14, 15, 16, 17, 18, 19, 20, 25, 30, 35, 40, 45, 50, 55, 60, 70 or 80 nucleosides. As used herein, a “telomere-specific probe” hybridizes to a telomere within a DNA molecule under moderate or high stringency, preferably high stringency. Typically, the telomere-specific probe has 85%, 86%, 87%, 89%, 90%, 91%, 92%, 93%, 94%, 95%, 96%, 97%, 98%, 99% or 100% homology to a telomere sequence. It is understood that, given the repetitive nature of the sequence of telomeres and thus the presence of multiple copies of the same target sequence, a telomere-specific probe can hybridize to multiple positions within the telomere.

The term “stretching” in relation to DNA refers to the process of elongating double-stranded DNA so as to minimize the amount of tertiary structure in the DNA, e.g. unfolding coiled DNA structures. Accordingly, the process of stretching DNA results in an increase in the physical length of a DNA fibre (e.g. in μm), while the sequence length (e.g. in kb) remains the same. For the purposes of the present invention, the process of stretching DNA involves elongating and depositing DNA fibres on to a support (e.g. a coverslip or microscope slide).

As used herein, “stretching rate” or “stretching factor” refers to the rate at which DNA fibres are stretched on to a surface of a support, such as a microscope slide or coverslip. The stretching rate is expressed as “sequence distance”/“physical distance”, e.g. kb/μm, and is also the conversion factor to convert a physical distance or length that is measured on stretched DNA (e.g. in μm) to a sequence distance or length (e.g. in kb). A “uniform” or “constant” stretching rate means that the rate at which one or more DNA fibres is stretched on to a surface of a support does not vary, or only varies by an amount that does not affect the reliable deduction of the sequence length from the measured physical length without the use of an internal control (i.e. a control of known sequence length). Thus, it is understood that a uniform or constant stretching rate can not be achieved by manually stretching DNA, i.e. stretching DNA without the aid of an apparatus or machine to control the stretching rate. Typically, a uniform or constant stretching rate is one that varies less than about 15%, 14%, 13%, 12%, 11%, 10%, 9%, 8%, 7%, 5%, 4%, 3%, 2% or 1% of the nominal stretching rate. Stretching DNA on to the surface of support at a uniform or constant rate typically produces essentially linear and parallel strands of DNA on the surface of the support. An exemplary method for stretching DNA at a uniform or constant rate is molecular combing, and in particular with the use of a molecular combing machine.

The terms “subject”, “individual” or “patient”, used interchangeably herein, refer to any animal subject, particularly a mammalian subject. By way of an illustrative example, suitable subjects are humans.

The term “telomere extension” refers to the process of elongation of a telomere by the addition of one or more nucleotides.

Those skilled in the art will appreciate that the aspects and embodiments described herein are susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features.

### 2. Methods for Assessment of Telomeres

Methods for assessing telomeres, including methods for measuring telomere length and methods for detecting telomere extension events and measuring telomere extensions, are described herein. The methods were developed to overcome one or more disadvantages of the previously-described methods, such as the previously-described methods for measuring telomere length.

As discussed above, the previously-described methods for assessing telomere length, including the commonly-used TRF, Flow-Fish, Q-PCR and STELA techniques, are either unable to facilitate measurements of absolute telomere length, are unable to facilitate measurements of individual telomere length, are inaccurate or inconsistent, and/or are not amenable to high-throughput formats. One rarely-used and rarely-described method for assessing telomere length (indeed, it is not even included in the reviews by Aubert et al. (Mutat Res. 2012, 730(1-2): 59-67) and Montpetit et al. (Nurs Res. 2014, 63(4): 289-299), is a fibre-FISH technique (Yan et al. The American Society of Human Genetics 54th Annual Meeting, Toronto, Canada, 2004; Wang et al. PLoS ONE 8(9): e75674). This technique manually stretches DNA fibres on to slides before hybridizing the fibres to a PNA probe that is specific for the telomeric repeats. The hybridized probe is then visualized under a microscope and the length of individual telomeres is calculated with reference to an internal control, e.g. a sequence of known length to which a specific probe has been hybridized. These internal controls are absolutely required because the DNA fibres are stretched on to the slide in an inconsistent manner with a variable rate. Even with these internal controls, the variability that can be present in the stretching rate from one end of a DNA fibre to the other means that the method lacks true reproducibility and consistency. The requirement for internal controls, the inconsistency even with these internal controls and the laborious nature of the method means that these fibre-FISH techniques are not suitable for clinical use, and in particular large-scale and high-throughput clinical platforms.

Advantageously, the methods of the present invention address one or more shortcomings of the previously-described methods. In particular, they facilitate the accurate measurement of the absolute length (i.e. not relative length) of individual telomeres in a sample. Moreover, the methods for measuring telomere length can be used with fresh, frozen or fixed cells of any cell cycle phase, and are conducive to high-throughput analysis. Thus, in some embodiments, the methods of the present invention can be performed, at least in part, by an automated system that facilitates high-throughput analysis of DNA samples.

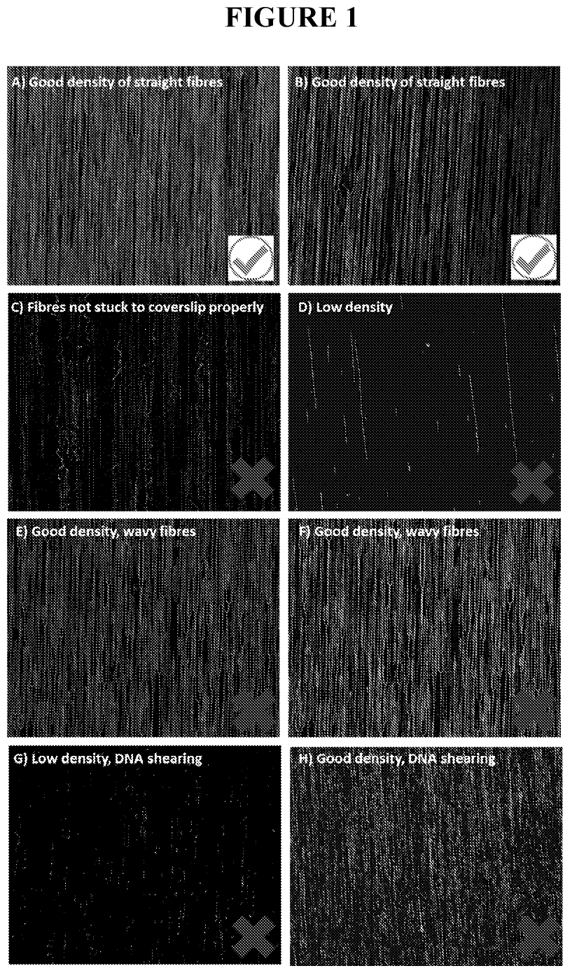

In their broadest sense, the methods of the present invention are performed by stretching DNA fibres on to a support at a uniform stretching rate (i.e. a uniform speed or uniform stretching factor). This can produce essentially linear and parallel strands of stretched DNA, as shown in FIGS. 1A and B. Telomeres are then detected by hybridizing the stretched DNA with a telomere-specific probe that binds along the length of the telomere and then detecting, such as visualizing, the probe-DNA hybrid (e.g. under a microscope). Depending upon the particular method and application, as described in greater detail below, the length of the entire portion of individual telomeres or the length of part of individual telomeres (e.g. individual telomere extensions) can be determined. This can be achieved by simply visualizing and measuring the length of the probe-DNA hybrid. In other embodiments, the intensity of a detectable signal (e.g. a fluorescent signal) from the hybridized probes is used to determine the length of the telomere or telomere extension.

The use of a constant stretching rate to uniformly stretch DNA fibres on to a surface of a support provides consistency not only within a sample or test (i.e. between DNA fibres on a single surface and within a single DNA fibre), but between samples or tests (i.e. between DNA fibres on different supports, whether the DNA fibres are from the same or different samples, or are processed at the same or different time). Accordingly, there is no need for internal controls for each support or each sample to take into account any variability in the stretching rate between DNA fibres and even within a DNA fibre. Accordingly, in certain embodiments, the methods of the present invention exclude controls that determine variation of DNA stretching rate between DNA fibres and/or within a single DNA fibre.

2.1 DNA Stretching

The DNA fibres can be stretched on to the desired surface using any technique that facilitates a constant stretching rate. Such techniques include, for example, molecular combing, molecular threading (Payne et al. PLoS ONE 2013, 8(7): e69058) and acoustic force spectroscopy (AFS; Gerrit Sitters et al. Nature Methods 2015, 12:47-50).

One exemplary technique is molecular combing, which is a simple and reproducible method for stretching DNA fibres (see e.g. Bensimon et al. Science 1994; 265:2096-2098; Bensimon et al. Phys. Rev. Lett. 1995; 74:4754-4757; Lebofsky and Bensimon, Brief Funct Genomic Proteomic. 2003, 1(4):385-96; WO 1995/22056; WO 1995/21939; WO 2008/028931 and U.S. Pat. No. 6,303,296). Briefly, a chemically-modified, hydrophobic support is dipped into a buffered solution containing DNA fibres, which then bind to the surface of the support at one or both extremities of the fibre in a pH-dependent manner. Suitable hydrophobic supports for molecular combing include polymers such as polystyrene (PS), polymethylmethacrylate (PMMA) and polycarbonate, as well as glass treated with hydrophobic silanes. For the purposes of the methods of the present claims, the support is typically a silanized glass support, such as a microscope slide or coverslip, to which the DNA attaches irreversibly (see e.g. Labit et al. BioTechniques, 2008; 45: 649-658). In a particular embodiment, the glass support is coated with vinyl silane.

The support is removed from the solution with a slow and constant speed so that the receding meniscus stretches the anchored DNA fibres on to the support with a constant perpendicular force. The force of the receding meniscus is insufficient to break either the DNA extremity-surface interaction or covalent bonds within the DNA molecule. However, the receding meniscus exerts sufficient force to stretch DNA from its random-coil conformation to approximately 150 percent of its molecular contour length. These stretched DNA fibres are irreversibly fixed on to the support and are aligned in a linear and parallel manner across the surface of the support, which facilitates easy visualization and assessment of the fibres. The use of a constant speed to remove the support results in a constant stretching factor, which, as described above, eliminates the need for internal controls.

Molecular combing can be performed using any apparatus or system designed or adapted for that use, and such apparatus and systems have been described elsewhere (see e.g. Kaykov et al. Sci Rep. 2016; 6: 19636; Gallo et al. Cold Spring Harb Protoc, 2016, pdb.prot085118). In a particular embodiment, the FiberComb® Molecular Combing System (Genomic Vision) is used. The FiberComb® Molecular Combing System contains two sub-assemblies which operate simultaneously. The operation of each sub-assembly is controlled and is monitored by a microcontroller. Each sub-assembly is able to hold one or two 22 mm square glass coverslips (e.g. glass coverslips coated with vinyl silane), submerge the coverslips into the disposable DNA reservoirs containing the DNA solution being analyzed, maintain the coverslips in the solution for the required duration and then withdraw the coverslips from the reservoir at a slow, constant speed (i.e. the stretching rate).

While any constant stretching rate can be used in the methods provided herein, typically the stretching rate is between 0.1 kb/μm and 10 kb/μm, between 0.5 kb/μm and 5 kb/μm, or between 1 kb/μm and 3 kb/μm. For example, the stretching rate can be 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 or 10 kb/μm. In particular embodiments, the stretching rate is 1.5 to 2.5 kb/μm, such as 1.5, 1.6, 1.7, 1.8, 1.9, 2.0, 2.1, 2.2, 2.3, 2.4, 2.5 kb/μm. In one example, the stretching rate is 2.0 kb/μm.

2.2 Telomere-Specific Probe and Hybridization

Following stretching of the DNA fibres at a constant rate on to the surface of the support, the DNA is hybridized to a telomere-specific probe to facilitate detection of the telomere. Exemplary telomere-specific probes hybridize to telomere repeat sequences to form a probe-DNA hybrid that contains many probes along the length of the telomere. Thus, in one embodiment, the probe hybridizes to the TTAGGG (SEQ ID NO:1) repeat sequence in vertebrate telomeres (including human telomeres), or the reverse complementary CCCTAA (SEQ ID NO:2) sequence. Also envisioned are probes that bind to telomeres of other species, such as the TTTAGGG (SEQ ID NO:3), TTTTTTAGGG (SEQ ID NO:4), or CTCGGTTATGGG (SEQ ID NO:5) telomere repeat sequence in some higher plants, the TTTTAGGG (SEQ ID NO:6) telomere repeat sequence in some algae, or the TTAGG (SEQ ID NO:7) telomere repeat sequence in some insects, or their reverse complementary sequences. Thus, in a particular embodiment, where the methods of the present invention are used to assess telomeres in DNA from a vertebrate such as a human, the probes comprise the sequence CCCTAA (SEQ ID NO:2) such that they bind to the TTAGGG (SEQ ID NO:1) repeat sequence. Such probes may comprise two or more repeats of this sequence so as to increase specificity (e.g. CCCTAACCCTAA (SEQ ID NO:8) or CCCTAACCCTAACCCTAA (SEQ ID NO:9). In a further embodiment, the probes comprise the sequence TTAGGG (SEQ ID NO:1) such that they bind to the reverse complementary CCCTAA (SEQ ID NO:2) repeat sequence in vertebrates. Typically, such probes comprise two or more repeats of this sequence so as to increase specificity (e.g. TTAGGGTTAGGG (SEQ ID NO:10) or TTAGGGTTAGGGTTAGGG (SEQ ID NO: 11)). In further embodiments, the probes hybridize to telomere variant repeats. These variant repeats differ from the canonical sequence at one or two nucleotide positions. Exemplary variant repeats in human genomic DNA include TCAGGG (SEQ ID NO:12), TGAGGG (SEQ ID NO:13), and TAAGGG (SEQ ID NO:14) and their reverse complement sequences, CCCTGA (SEQ ID NO:15), CCCTCA (SEQ ID NO:16), and CCCTTA (SEQ ID NO:17). Accordingly, the probes may comprise the sequence set forth in any one of SEQ ID NOs: 12-17. Variant repeats are abundant in the proximal (centromeric) end of the telomere. Accordingly, the use of such probes may provide additional information on the directionality of the telomere.

The telomere-specific probes may comprise, for example, DNA and/or RNA, and/or analogues thereof, including peptide nucleic acid (PNA), locked nucleic acid (LNA), unlocked nucleic acid, (UNA) and triazole-linked DNA. Exemplary probes for use in the methods described herein are telomere-specific PNA probes. PNA probes are synthetic DNA mimics, with the sugar phosphate backbone of the DNA helix replaced by an uncharged, structurally-homomorphous, pseudopeptide backbone. PNA probes generally have improved properties compared to DNA probes, including improved binding capacity, higher specificity, improved hybridization efficiency and hybridization stability. As such, they are particularly useful in fluorescent in situ hybridization (FISH) applications, even at low concentrations, with strong and specific hybridization occurring in just a couple of hours.

The probes may comprise a detectable label to facilitate detection, e.g. by visualization. In particular embodiments, the probes comprise a fluorescent label to facilitate detection using a fluorescent microscope, such that the method of the invention utilizes fluorescence in situ hybridization (FISH) to assess the telomeres. Fluorophores of different colors may be chosen such that the telomere-specific probe can be visualized and distinguished from other labelling (e.g. from DNA counterstaining). Non-limiting examples of fluorophores include 7-amino-4-methylcoumarin-3-acetic acid (AMCA), Alexa Fluor 350; Alexa Fluor 405; Alexa Fluor 488; Alexa Fluor 532; Alexa Fluor 546; Alexa Fluor 555; Alexa Fluor 568; Alexa Fluor 594; Alexa Fluor 647; Alexa Fluor 680; Alexa Fluor 750; BODIPY FL; Coumarin; Cy3; Cy5; Fluorescein (FITC); Oregon Green; Pacific Blue; Pacific Green; Pacific Orange; rhodamine; tetramethylrhodamine (TRITC); carboxytetramethylrhodamine (TAMRA) and Texas Red.

The telomere-specific probes can be labelled using any method known in the art. The label may be attached to the probe prior to or after hybridization of the probe with the DNA fibres. Direct labels are detectable labels that are directly attached to or incorporated into the probe prior to hybridization. In contrast, indirect labels are attached to the probe after hybridization. In some examples, the indirect label is attached to a binding moiety that has been attached to the probe prior to the hybridization. Thus, for example, the probe may be biotinylated before the hybridization. After hybridization, an avidin-conjugated fluorophore will bind the biotin bearing hybrid duplexes providing a label that is easily detected.

The labels can be attached directly or through a linker moiety. In general, the site of label or linker-label attachment is not limited to any specific position. For example, a label may be attached to a nucleoside, nucleotide, or analogue thereof at any position that does not interfere with detection or hybridization. For example, certain Label-ON Reagents from Clontech (Palo Alto, Calif.) provide for labeling interspersed throughout the phosphate backbone of an oligonucleotide and for terminal labeling at the 3′ and 5′ ends. For a review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)). Fluorescently-labelled telomere-specific probes for use in FISH applications are known in the art and are commercially available.

The telomere-specific probe is hybridized to the stretched DNA under any condition effective to facilitate binding and formation of the probe-DNA hybrid, after which the DNA is subsequently washed to remove unbound probe. Suitable conditions for hybridization and washing are well known to those in the art and can be readily determined, based in part on the melting temperature (Tₘ) of the probe. Typically, the probe is hybridized under medium or high stringency conditions to reduce background or non-specific binding. Where commercially-available probes are used, the manufacturer's instructions for hybridizing the probes to the DNA may be followed. Exemplary conditions for hybridizing a telomere-specific probe are detailed below in Example 1.

2.3 DNA Counterstaining

In addition to being contacted with a telomere-specific probe to facilitate assessment of the telomere, the DNA may also be contacted with a DNA counterstain to visualize the entire DNA fibre, which may assist in visualizing the telomere. Exemplary DNA counterstains include, but are not limited to, intercalating dyes such as phenanthridines and acridines (e.g., ethidium bromide, propidium iodide, hexidium iodide, dihydroethidium, ethidium homodimer-1 and -2, ethidium monoazide, and ACMA); minor grove binders such as indoles and imidazoles (e.g., Hoechst 33258, Hoechst 33342, Hoechst 34580 and DAPI); and miscellaneous nucleic acid stains such as acridine orange (also capable of intercalating), 7-AAD, actinomycin D, LDS75 1, and hydroxystilbamidine. All of the aforementioned nucleic acid stains are commercially available from suppliers such as Molecular Probes, Inc. Still other examples of nucleic acid stains include the following dyes from Molecular Probes: cyanine dyes such as SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red). As would be understood by the skilled person, the counterstain is selected such that the labelled DNA can be differentiated from the telomere-specific probe, e.g. the counterstain and labelled telomere-specific probe have different emission characteristics and can be visualised as different colours under a fluorescent microscope.

2.4 Detection

Following hybridization with the telomere-specific probe and optional DNA counterstaining, DNA fibres on the support, and in particular the probe-DNA hybrids, are then detected. The process of detection will depend on the type of detectable label on the probe and, where used, the type of DNA counterstain, and can easily be determined by those skilled in the art. Typically, the probe-DNA hybrids are visualized under a microscope. For example, where fluorescently-labelled telomere-specific probes are used, a fluorescent microscope is used to visualize DNA fibres and detect telomeres. The telomeres are assessed as described in greater detail below to measure their length, to detect a telomere extension event and/or to measure the length of telomere extension. This can be done manually or with the use of software and/or a specialised system, and can optionally involve images being taken of the DNA fibres by a camera connected to the microscope. For example, an imager or camera attached to the microscope, and associated software, can be used to detect and measure the length of a telomere or telomere extension event. In another example, a system such as the FibreVision® scanner (Genomic Vision) can be used to acquire high resolution images of the whole surface of a coverslip onto which DNA has been stretched and hybridized with the telomere-specific probe. Additional systems for detecting, visualizing and measuring the length of a telomere are described in more detail below.

2.5 Measurement of Telomere Length

The methods of the present invention include methods for measuring the length of a telomere. As detailed above, the methods include stretching genomic DNA from a cell on to a surface of a support (e.g. a silanized glass coverslip or slide) at a constant stretching rate before hybridizing a telomere-specific probe to the DNA to form a probe-DNA hybrid. By virtue of the probe binding along the full length of the telomere, the length of the telomere is directly proportional to the number of probes that bind thereto, allowing for measurement of the length of the telomere.

Measurement of the length of the telomere can be performed by visualizing the probe-DNA hybrid, such as under a fluorescent microscope, and directly measuring the physical length of the probe-DNA hybrid on the support, e.g. in μm. This length can then be converted to the sequence length (e.g. in kb) by taking the stretching factor into account, i.e. multiplying the length in μm by the stretching factor. The microscope may be fitted with a camera and images of the probe-DNA hybrid can be taken. In some instances, software associated with the microscope is used to analyze the images and then determine the physical length of the probe-DNA hybrid, and/or convert the physical length to sequence length (e.g. μm to kb).

Measurement of the length of the telomere can also be performed by detecting the intensity of a detectable signal from the telomere-specific probe that has hybridized to the DNA. For example, where the telomere-specific probe is fluorescently labelled, the intensity of the fluorescent signal can be used to measure the length of the telomere. The intensity of the signal will be directly related to the number of probes that bind to the telomere, and thus directly related to the length of the telomere. A calibration curve, in which the fluorescence intensity for a particular probe is plotted against telomeres of known length and/or the number of probes in a sample, can be used to convert the observed fluorescence intensity to telomere length.

As noted above, the use of a constant stretching rate to uniformly stretch the individual DNA fibres on to a surface of a support provides consistency not only within a sample but also between samples and between different analyses (e.g. between analyses performed at different times). There is therefore consistency between DNA fibres from the same sample on the same support, from the same sample but on different supports, and from different samples on different supports. Accordingly, there is no need for internal controls for each support, sample or analyses to take into account any variability in the stretching rate between DNA fibres or even within a DNA fibre: the absolute length of a telomere can be measured directly, accurately and reproducibly.

Notwithstanding this, in some applications an internal control may be used, such as to confirm that the DNA is being stretched at the desired, uniform rate. For example, where a molecular combing machine is utilized for the present methods, an internal control may be included on one support each time the machine is turned on or for each batch of samples being analyzed to confirm that the machine is correctly calibrated and is performing as expected. Internal controls can include, for example, exogenous DNA molecules of known length (i.e. DNA molecules that are not part of the genomic DNA being analyzed), or endogenous DNA (i.e. DNA within the genomic DNA being analyzed). Where exogenous DNA molecules are used, these can be stretched on to the surface of a support that does not contain any genomic DNA being analyzed, and then visualized using a DNA counterstain (e.g. YOYO-1), as described below in Example 1. Alternatively, the exogenous DNA molecules may be synthetic molecules to which a detectable probe can hybridize along the length so as to facilitate visualization. Endogenous DNA controls include, for example, regions to which a labelled probe of a known size can bind. For example, subtelomere probes of known size can be hybridized to the stretched DNA and be used as an internal control.

The methods provided herein for measuring the length of a telomere can advantageously be performed using genomic DNA from fixed, frozen or fresh cells. Moreover, the cells can be of any cell type, including cells in any biological sample, including any tissue sample or bodily fluid (e.g. blood), from which genomic DNA can be extracted. Methods for extracting genomic DNA from cells are well known in the art and any such methods can be used herein.

2.6 Detection and/or Measurement of Telomere Extension

The methods of the present invention also include methods for detecting and optionally measuring telomere extension. These methods involve contacting a cell with a nucleotide analogue under conditions sufficient for DNA synthesis, such that the nucleotide analogue is incorporated into any newly-synthesized DNA, including any newly-synthesised telomeric DNA. The genomic DNA is then isolated from the cell, stretched on to a surface of a support at a uniform stretching rate and hybridized with a telomere-specific probe to produce a probe-DNA hybrid, as described above.

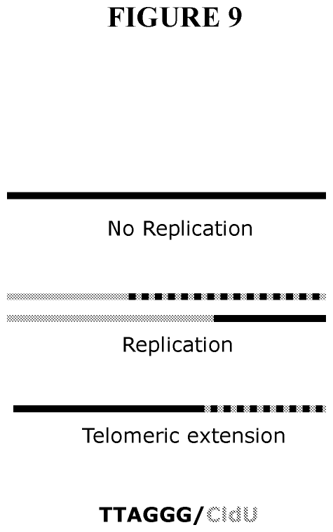

Any “pre-existing” telomeric DNA will only be hybridized to the probe, while newly-synthesized telomeric DNA (i.e. resulting from DNA replication or extension) will be hybridized to the telomere-specific probe and will also contain newly-incorporated analogue. The synthesis of a telomere by extension can be distinguished from the synthesis of a telomere by replication by the fact that the newly-extended telomere will be directly adjacent to pre-existing telomere (see FIG. 9). Thus, telomere extension is detected when a probe-DNA hybrid (which is indicative of telomeric DNA) also contains newly-incorporated nucleotide analogue (which is indicative of DNA synthesis/extension) and is adjacent to a probe-DNA hybrid that does not contain incorporated nucleotide analogue (which is indicative of the “pre-existing” telomere or the telomere prior to extension). Optionally, the length of each telomere extension and or each pre-existing telomere can then also be determined as described above in section 2.5.

Suitable nucleotide analogues include, for example, halogenated thymidine analogs such as bromodeoxyuridine (BrdU), chlorodeoxyuridine (CldU) iododeoxyuridine (IdU), and ethynyldeoxyuridine (EdU) which have been widely used to detect DNA synthesis and replication (see e.g. Yokochi and Gilbert, Curr Protoc Cell Biol. 2007 Chapter 22:Unit 22; and Salic and Mitchinson, PNAS 2008, 105:2415-2420). These analogues are typically not directly labelled with a visually-detectable label prior to incorporation into the DNA, so a detectable label is applied after incorporation into the DNA. This can be achieved, for example, with labelled antibodies, such as fluorescently-labelled antibodies, which are specific for the analogues. In other examples, such as when EdU is used as the nucleotide analogue, fluorescent azides are used in a Cu(I)-catalyzed [3+2] cycloaddition (“click” chemistry) (see e.g. Salic and Mitchinson, PNAS 2008, 105:2415-2420). Fluorescent nucleotide analogues can also be employed in the present methods. Fluorescent nucleotide analogues include, but are not limited to, 2-aminopurine (2AP), pyrrolo-C (PyC), 1,3-diaza-2-oxophenothiazine (tC), 1,3-diaza-2-oxophenoxazine (tC°) and 7-nitro-1,3-diaza-2-oxophenothiazine (tCₙᵢₜᵣₒ).

Cells can be contacted with the nucleotide analogue in vitro, in vivo or ex vivo, and suitable conditions that facilitate incorporation of the nucleotide analogue into newly-synthesised DNA in cells are well known in the art. For example, the incorporation of thymidine analogues into the DNA of a variety of cells (so-called thymidine incorporation assays) has been widely-reported for many decades. Any viable cell capable of DNA synthesis can be used, including cultured cell lines and primary cells, whether fresh or frozen and then thawed. The cells may be inherently capable of telomere extension, such as by utilizing the ALT pathway (e.g. VA-13, U-2 OS, JFCF-6/T.1R, JFCF-6/T.1M, IIICF/c, GM847 and AG11395 cells) or by expressing active telomerase components (e.g. JFCF-6/T.1F, GM639, HeLa, HT1080 (wild-type hTR and hTR TCA cells). In other examples, the cells are capable of DNA synthesis but are essentially incapable of telomere extension without the addition of an agent that promotes telomere extension.

Thus, also envisioned are methods in which the cells are contacted with an agent that does or that may promote or inhibit telomere extension. The cells are contacted with the agent before, after or simultaneously to contacting the cells with the nucleotide analogue. The occurrence of any telomere extension, and/or the length of that telomere extension, can then be assessed. Typically, the occurrence of any telomere extension, and/or the length of that telomere extension in the presence of the agent is compared to the telomere extension observed in the absence of the agent, so to determine whether the agent is able to promote or inhibit telomere extension and to what degree. The agent may comprise, for example, a nucleic acid (e.g. an antisense oligonucleotide, siRNA, miRNA, or nucleic acid encoding any one of these or a protein), a small molecule, a peptide or a polypeptide, or any combination thereof.

### 3. Kits and Systems

All the essential materials and reagents required for measuring the length of a telomere, detecting a telomere extension event and/or measuring a telomere extension event, and related methods as described herein, may be assembled together in a kit. For example, the kit may comprise a telomere-specific probe, such as a labelled telomere-specific PNA probe (such as any described above or known in the art), and optionally a DNA counterstain, such as YOYO-1 (or any other counterstain known in the art). When the methods of the present invention include first isolating the genomic nucleic acid to be analyzed, kits comprising reagents to facilitate that isolation are also envisioned. Such reagents can include, for example, one or more buffers, such as one or extraction buffers, wash buffers and/or proteinase buffers. Kits suitable for performing the methods for detecting and/or measuring telomere extension may also comprise a nucleoside analogue. The kits will also generally comprise, in suitable means, distinct containers for each individual reagent. The kits can also feature various devices, and/or printed instructions for using the kit.

In some embodiments, the methods described generally herein are performed, at least in part, by a processing system, such as a suitably programmed computer system. A stand-alone computer, with the microprocessor executing applications software allowing all or a part of the above-described methods to be performed, may be used. Alternatively, the methods can be performed, at least in part, by one or more processing systems operating as part of a distributed architecture. For example, one or more processing systems with associated software can be used to effect the stretching of the DNA on to the surface of a support as described above, the hybridization of the stretched DNA fibres to the telomere-specific probe, the imaging of the support containing the DNA fibres and hybridized probe, and/or the measurement of the length of a telomere. In some examples, commands inputted to the processing system by a user assists the processing system.

In one example, a processing system includes at least one microprocessor, a memory, an input/output device, such as a keyboard and/or display, and an external interface, interconnected via a bus. The external interface can be utilized for connecting the processing system to peripheral devices, such as a communications network, database, or storage devices. The microprocessor can execute instructions in the form of applications software stored in the memory to allow all or a part of the methods of the present invention to be performed, as well as to perform any other required processes, such as communicating with the computer systems. The applications software may include one or more software modules, and may be executed in a suitable execution environment, such as an operating system environment, or the like.

Particular exemplary processing systems, and methods that utilize those systems, are described in more detail in Example 3, below.

### 4. Applications

The methods described herein to measure the length of telomeres, detect telomere extension and/or measure the length of a telomere extension can be used in a variety of diagnostic, prognostic and research applications.

The methods described herein for measuring the length of a telomere can be used to diagnose a disease, determine the risk of developing a disease and/or determine the prognosis of a disease, where telomere length has been associated with that disease (e.g. short telomere or long telomere). Telomere length has been reported as a diagnostic or prognostic indicator for a range of conditions, disorders and diseases. For example, telomere syndromes, or telomeropathies, are genetic diseases caused by defects in the proteins required for telomere maintenance (for review, see e.g. Holohan et al. J Cell Biol. 2014, 205 (3):289-299 and Armanious and Blackburn, Nat Rev Genet. 2012, 13(10):693-704.). The underlying mutations are in genes encoding a range of proteins, but in all instances, subjects present with short or dysfunctional telomeres. As such, an assessment of telomere length or telomere sequence content can be used to help diagnose a telomere syndrome in a subject. Such telomere syndromes include, but are not limited to, Dyskeratosis Congenita, Hoyeraal-Hreidersson syndrome, Revesz syndrome, Coats Plus syndrome. Mutations in the genes associated with telomere maintenance, leading to shortened telomeres, often result in idiopathic pulmonary fibrosis, liver fibrosis, bone marrow failure and aplastic anemia. Other syndromes that are associated with short telomeres but are not the result of a mutation in proteins involved in telomere machinery (sometime referred to as secondary telomeropathies) include, for example, Fanconi anemia, Rothmund-Thomson syndrome, and ICF (immunodeficiency, centromeric region instability, and facial anomalies syndrome type I).

Telomere length has also been linked with other conditions, including cancer. Cancers for which disease risk or prognosis has been linked to telomere length include, but are not limited to, prostate cancer, head and neck cancer, gastrointestinal cancer (including colorectal cancer, gastric cancer etc), lung cancer, acute myeloid leukemia, acute lymphoblastic leukaemia, cervical cancer, bladder cancer, lymphoma and skin cancer (including melanoma). The relationship between telomere length and various cancers appears quite complex, with long, short and/or variable telomere length, and in particular cells, being associated with cancer risk or prognosis. For example, the combination of variable telomere length in prostate cancer cells and shorter telomere length in stromal cells strongly correlates with progression to prostate cancer metastasis and cancer death (Heaphy et al. Cancer Discov. 2013 October; 3(10): 1130-1141). In another example, both very long and very short telomere lengths have been associated with the risk of developing colorectal cancer (Cui et al. Cancer Epidemiol Biomarkers Prev. 2012, 21, 1807-13). In addition, cancer patients with short telomeres are prone to treatment side effects. For instance, associations between short telomere length and (i) liver toxicities from chemotherapy, (ii) higher rates of fungal infection, and (iii) increased need for platelet transfusion requirements, have been identified in childhood cancer patients. Thus, the methods described herein to measure telomere length can be used to determine the risk of a subject developing cancer, and/or the prognosis of a subject with cancer (and thus suitable treatment protocols based on that risk or prognosis).

Additionally, short telomeres have been associated with cardiovascular disease (Haycock et al. BMJ 2014, 349:g4227; Yeh and Wang, Genes, 2016, 7(9):58) and are possible indicators and risk factors for other age-related diseases, including, for example, diabetes, Alzheimer's disease and hypertension, as well as related functions or presentations, such as cognition, bone loss and longevity (for review, see e.g. Muzumdar and Atzmon Telomere Length and Aging, in Reviews on Selected Topics of Telomere Biology, Radhika Muzumdar and Gil Atzmon, Telomere Length and Aging, Reviews on Selected Topics of Telomere Biology, Dr. Bibo Li (Ed.), InTech, 2012, 3-29).

In addition to being used to help diagnose, determine the risk of, and/or determine the prognosis of, any one of the diseases described above, the methods of the present invention can be used to help determine (or help more accurately determine) the association between telomere length and any given disease or condition, or lifestyle factor (e.g. diet, exercise, etc.). The increasing interest in accurately determining the association between various diseases or conditions and telomere length means that there is an increased need for accurate, reliable and convenient methods for measuring telomere length. This may be particularly true for diseases where the association between disease and telomere length is complex and more difficult to determine (e.g. is not a linear association, is cell-dependent, etc.) and requires studies involving large sample and data sets. In some embodiments, the methods described herein can meet this need.

The methods described herein for detecting telomere extension and/or detecting the length of telomere extension can be used in a variety of research, pre-clinical or clinical studies, such as, for example, studies to identify or evaluate agents that promote or inhibit telomere extension for research or therapeutic purposes. For example, cells can be exposed to an agent prior to, simultaneously, and/or after the cells are contacted with the nucleotide analogue, and the presence and/or extent of telomere extension can be determined as described above and compared with telomere extension in the absence of the agent. Agents that promote telomere extension can include agents that provide exogenous telomerase activity (e.g. one or more components of telomerase or the nucleic acid encoding telomerase), agents that re-activate silenced endogenous telomerase (e.g. histone deacetylase inhibitors and estrogen receptor agonists), and agents that further activate residual telomerase activity, while agents that inhibit telomere extension can include agents that inhibit telomerase activity, including but not limited to, antisense oligonucleotides and small molecule inhibitors (for review, see e.g. Jager and Walter, Genes (Basel). 2016 July; 7(7): 39).

In order that the invention may be readily understood and put into practical effect, particular preferred embodiments will now be described by way of the following non-limiting examples.

### EXAMPLES

### Example 1

### Measurement of Teleomere Length Using Telomere Fibre-Fish (TFF)

Telomere length can be assessed using a variety of assays, each with their own limitations. Terminal restriction fragment (TRF) is perhaps the “gold standard”, although qPCR methods and Flow-FISH techniques are also widely used. None of these techniques provides the ability to measure individual telomere lengths and absolute telomere length in a high throughput manner. One other rarely-described technique, fibre-FISH, does facilitate the measurement of individual telomere lengths. However, it lacks reproducibility, requires an internal control and is not amenable to high throughput analysis of samples. Telomere Fibre-FISH (TFF) was therefore developed as an alternative and improved technique for measuring the absolute length of individual telomeres in a format conducive to high-throughput analysis. This method involves stretching DNA fibres on to a coverslip at a constant rate, such as by using a molecular combing machine, before hybridization to a labelled telomere-specific probe that allows visualization of individual telomeres and subsequent measurement of their lengths.

### A. Comparison of TFF and Previous Fibre-FISH Methods

A side-by-side comparison of TFF and a previously-described fibre-FISH method, in which DNA fibres are stretched on to a slide manually, was performed. Initially, a comparison of the DNA fibre extension techniques used in TFF and the previously-described fibre-FISH method was performed using control DNA fragments of defined length (10 kb). After being stretched on to the coverslips, as described below, these 10 kb fragments were stained with the YOYO-1 DNA counterstain and visualized as described below to assess the consistency, reproducibility and accuracy of the DNA extension techniques. Following this initial analysis, the TFF and fibre-FISH methods were used to measure telomere lengths in U-2 OS cells, an ALT cancer cell line that has highly heterogeneous telomere lengths.

### Methods

### Telomere Fibre-FISH

TFF was performed by first culturing cells in a T75 flask until at approximately 70% confluency, then harvesting the cells by aspirating media, rinsing in PBS and adding 1 ml of trypsin to the flask and incubating at 37° C. for 5 minutes. The cells were removed and the trypsin inactivated by adding an additional 9 ml of media. The cells were then removed and washed twice with PBS solution. Samples containing 1 million cells in 45 μL PBS were prepared for each subsequent agarose plug. The samples were placed in a 50° C. water bath, as were agarose solutions prepared using low melting agarose. The cell samples and agarose solutions were incubated for 10 to 30 seconds before agarose was added to the cell samples at 1:1 ratio and mixed by pipetting up and down gently. The resulting solution (90 μL) for each sample was pipetted into a plug (BioRad) and the plug was allowed to set for 30 min at 4° C. Plugs can be stored for up to 3 weeks at 4° C. wrapped tightly in parafilm and foil as needed.

ESP solutions containing EDTA, Sarcosyl and Proteinase K were prepared (200 μl 0.5M EDTA pH 8.0, 25 μL 10% (w/v) Sarcosyl/0.5M EDTA, 50 μl 20 mg/ml Proteinase K, for each plug) and added to each solidified plug in a 15 mL tube. The tubes were incubated at 50° C. overnight in a water bath (with no shaking in order to avoid DNA shearing). The ESP buffer was then discarded, using a gauze on the top of the tube to retain the plug in the tube. The tube was filled with Tris/EDTA buffer (TE: 10 mM TRIS-HCl, 1 mM EDTA, pH 8) and incubated for 1 hour at room temperature in rotation to wash the plug, a process that was repeated 3 times, with the fourth wash being for 3 hrs. Each washed plug was added to 1 ml 0.5M MES, pH=5.5, and incubated at 68° C. using a thermal-dry-block for 20 min. The tubes were then quickly transferred to a 42° C. thermal-dry-block and incubated for 10 min, after which 2 μL of Agarase (at room temperature) was added to the DNA solution. After incubation at 42° C. overnight, the DNA solution was gently poured into 800 μL MES buffer pH=5.5.

DNA was combed onto a silanized coverslip (Genomic Vision) using the Genomic Vision FiberComb Molecular Combing System. The coverslips with combed DNA were baked at 60° C. for 1 hr before being cooled to room temperature (protected from light).

The quality of DNA fibres was checked on one representative coverslip for each cell sample using a YOYO-1 counterstain on the coverslip, where 100 μL of 150 nM YOYO-1 (Invitrogen) in Blockaid (B-10710, Invitrogen) was placed on to a microscope slide in a humidity box before the combed coverslip was placed onto the YOYO solution and incubated for 20 min at 37° C. Following incubation, coverslips were placed in a 6-well plate with the stained DNA side facing upwards before being washed twice for 3 mins on shaker with YOYO washing solution (2×SSC, 1% (v/v) Tween-20 in milli-Q water). After a further rinse with water, the coverslips were removed and air dried on a tray in the dark at 37° C., with the stained side of the coverslip facing upwards.

Coverslips were mounted on a microscope slide using 7 μL of Prolong Gold mounting medium before being allowed to dry for 1-2 hrs. FIG. 1 shows the desired combing that results in a good density of straight fibres (rather than, for example low density of fibres, wavy fibres and/or DNA shearing).

Once the quality of the fibres was confirmed, samples for assessing telomere length were processed further. The combed coverslips were baked for 4 hours at 60° C. before being cooled to room temperature. Each coverslip was then placed into a well of a 6 well plate before being fixed for 5 minutes with 4% formaldehyde in PBS (5 mL of solution per well, removed by aspiration after completion of incubation). Optionally, a denaturation/fixation step was performed instead. This can increase subsequent telomere probe binding, although will also reduce YOYO-1 staining, thus making identification of telomeres more difficult. In this optional step, the DNA on the coverslips was denatured for 25 minutes in 0.5M NaOH, 0.1% b-mercaptoethanol in 70% ethanol (5 ml solution per well), before being fixed for 5 minutes in 250 μL of 10M NaOH with 5 μL b-mercaptoethanol.

After rinsing in water, the fixative was neutralized for 5 minutes in PBS. RNA was then removed by first aspirating the PBS then treating the coverslips for 30 minutes with RNAse A (100 μg/mL) in antibody dilution buffer (ABDIL: 20 mM Tris pH 7.5, 2% BSA, 0.2% Fish Gelatin, 150 mM NaCl, 0.1% Triton, 0.1% Sodium Azide). The RNAse solution was removed and the coverslips were rinsed in PBS before being dried with an ethanol dehydration series (3 min 70% ethanol, 2 min 90% ethanol, 2 min 100% ethanol). Coverslips were carefully removed from the 6-well plates and placed facing upwards on a drying tray before being dried in a 37° C. oven for 10 minutes.

Slides were prepared in a humidity box by adding 50 μL of 0.3 ng/mL solution of TAMRA-OO-(CCCTAA)₃ or TAMRA-OO-KKK(TTAGGG)₃ PNA telomere probe (Panagene) to each slide. The dry coverslips were then placed onto the PNA solution on the slides and the slides were incubated in the humidity box overnight. The coverslips were transferred to 6 well plates (stained surface facing upwards) and washed 4 times for 10 minutes each wash with 50 mM Tris pH 7.5, 150 mM NaCl, 0.08% Tween-20 on a shaker before a further wash with PBS for 5 minutes. The coverslips were rinsed with water and counterstained with 100 μL YOYO-1 (150 nM in Blockaid B-10710) for 20 minutes at 37° C. to help identify the DNA fibres. The coverslips were then transferred to a 6 well plate (with stained DNA side facing upwards) and washed twice for 3 minutes each wash with 2×SSC, 1% (v/v) Tween-20.

After rinsing with water, the coverslips were air dried on a tray in the dark at 37° C. for 20 minutes, with stained side of coverslip facing upwards. The coverslips were mounted on a microscope slide using 7 μL of Prolong Gold mounting medium (stained DNA fibres facing downwards).

### TFF Image Capture and Analysis

Slides were loaded onto the stage of a fluorescent microscope and visualized using the 63× eyepiece magnification to confirm staining of genomic DNA with YOYO-1 and staining of telomeric DNA with the PNA probe, and also to confirm that the fibres were of good quality (e.g. no shearing). Upon confirmation of these features, the images of the slides were captured with an Axio Imager Z2 (Zeiss) and analyzed using Zen software (Zeiss).

Images were captured either by taking individual 63× snapshots or by using the tiling function essentially according to the manufacture's instructions. Briefly, for individual snapshots, the slide was searched in an ordered fashion using the telomere-stained channel (TAMRA). Images in both stained channels (YOYO-1 and TAMRA) were then taken for each telomere identified. For the tiling function, the focus strategy included selection of ‘focus surface as start for autofocus’, the Alexa488 reference channel (or other filter used to capture YOYO-1 counterstain), and the “fixed Z-position”; and the range for the autofocus was adjusted to 30 μm. Multiple tiles were created with support points evenly dispersed within each tiled region. These support points were verified by adjusting the ‘Z-axis’ of each one to ensure the image was focused and clear. A 10% tile overlap was selected, and images were then taken.

Images were then opened with the Zen software for analysis. Telomeres were identified using the correct channel (TAMRA) and the absolute telomere length of each individual telomere was measured in microns using the line tool in the Zen software. This was then converted to kilobases (kb) by using the formula 1 μm=2 kb.

### Fibre-FISH

Fibre-FISH was performed by manually stretching DNA on to silanised microscope slides using the method described by Fransz et al. (Plant J (1996) 9, 421-430). Following DNA extension, the DNA fibres were treated and visualized as described previously in Wang et al. (PLOS ONE (2013) 8, e75674).

### Results

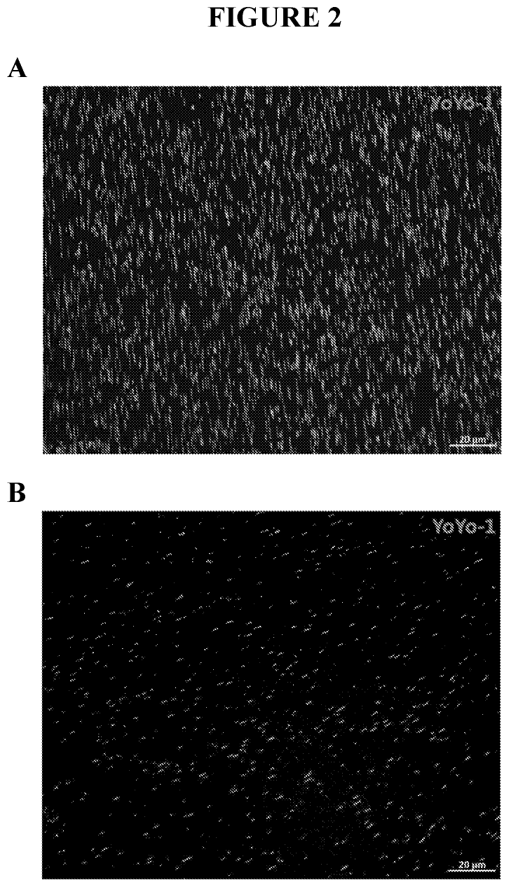

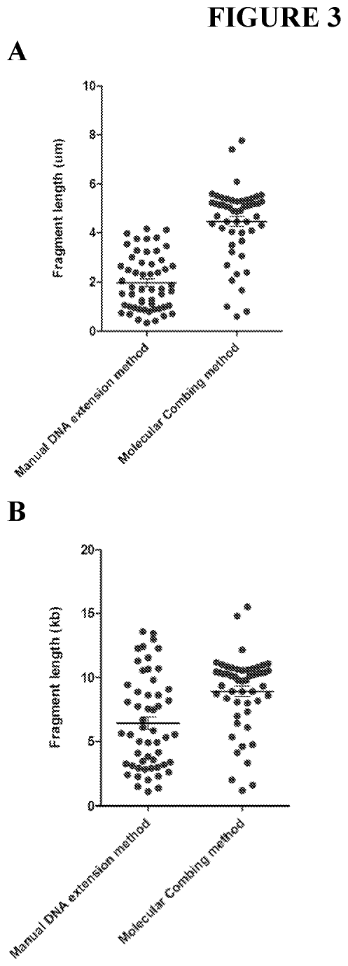

FIGS. 2 and 3 show the results of the initial comparison of the DNA fibre extension techniques used in TFF and the previously-described fibre-FISH. As can be clearly seen, manually stretching the 10 kb fragments (estimated to be at a rate of about 3.27 kb/μm as described in Fransz et al., 1996) was less reproducible, less consistent and less accurate (FIG. 2B) than the molecular combing technique used in TFF (FIG. 2A), resulting in a large range of observed telomere lengths that were, on average, significantly less than 10 kb (FIG. 3). In contrast, the molecular combing method used in TFF was far more consistent, with a significant portion of the fragments measured at about 10 kb (FIG. 3).

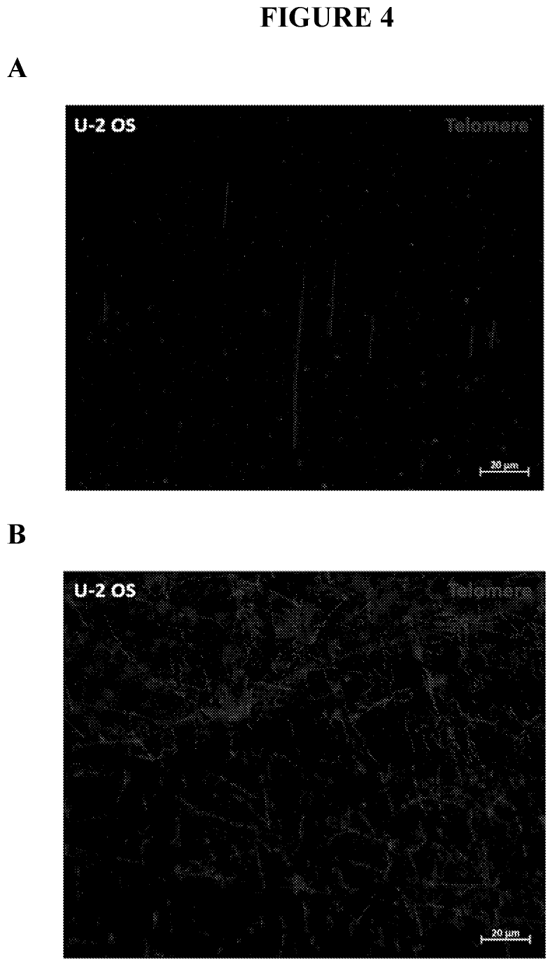

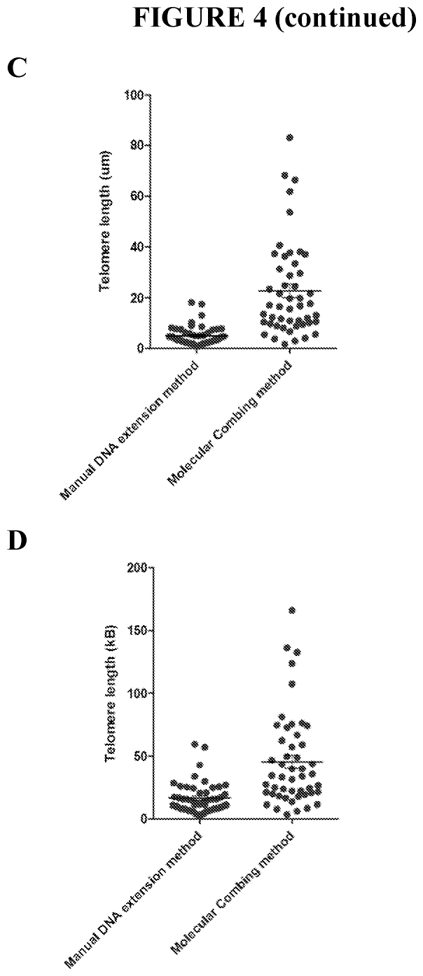

As shown in FIG. 4A, the stretched DNA fibres from U-2 OS cells obtained using TFF were linear and consistent, resulting in easy visualization of individual telomeres. In contrast, the DNA fibres obtained using the fibre-FISH method were not linear and were not stretched to the same extent as TFF or with the same consistency, making it difficult to visualize individual telomeres (FIG. 4B). When the lengths of individual telomeres were measured, the lengths as assessed using fibre-FISH were significantly shorter than those assessed using TFF (FIGS. 4C and D). TFF allowed for measurement of a larger range of telomeres, and in particular of larger telomeres.

### B. Comparison of TFF and TRF Techniques

A side-by-side comparison of telomere fibre-FISH (TFF) and terminal restriction fragment (TRF) length analysis, the current “gold-standard technique” for measuring telomere length, was performed. The lengths of telomeres from a range of ALT cells (i.e. cells that utilize the alternative lengthening of telomeres (ALT) pathway and Tel+ cells (i.e. cells in which telomerase is activated) were assessed in the first study, while the second study focused on the HT1080 telomerase-positive cell line.

### Methods

TFF was performed essentially as described above. Briefly, cells were isolated by trypsinization, embedded in agarose plugs to minimise DNA breaks, and subjected to proteinase K digestion. Molecular combing was performed using the Molecular Combing System (Genomic Vision S.A.) with a constant stretch factor of 2 kb/μm using vinyl silane coverslips (20×20 mm; Genomic Vision S.A.), according to the manufacturer's instructions. After combing, coverslips were dried for 4 hrs at 60° C. Quality and integrity of combed DNA fibres were checked using the YoYo-1 counterstain. Coverslips were denatured for 25 min in alkali-denaturing buffer (0.2M NaOH, 0.1% β-mercaptoethanol in 70% ethanol) and fixed by addition of 0.5% glutaraldehyde for 5 min. Telomeric DNA was visualized by hybridization with a TAMRA-OOKKK(TTAGGG)₃ PNA probe (Panagene). Telomere fibres were detected on a Zeiss Axio Imager microscope with ApoTome module and analyzed with Zen software (Zeiss).

TRF analysis was performed by first preparing terminal restriction fragments by HinfI and RsaI digestion of genomic DNA, and separating the fragments by pulsed-field gel electrophoresis. In brief, genomic DNA was digested with 4 U/μg HinfI and RsaI overnight at 37° C. The DNA was then ethanol precipitated and 2 μg was loaded on a 1% (wt/vol) agarose gel in 0.5×TBE. Pulsed-field gels were electrophoresed at 6 V for 14 hrs at 14° C. with an initial switch time of 1 sec and a final switch time of 6 sec. The gels were dried for 75 min at 60° C., denatured in 0.5 M NaOH/1.5 M NaCl for 1 h, and neutralized in 0.5 M Tris-HCl (pH 8.0)/1.5 M NaCl for 1 h. Gels were then rinsed in 2×SSC and prehybridized in Church buffer (250 mM sodium phosphate buffer, pH 7.2, 7% [wt/vol] SDS, 1% [wt/vol] BSA fraction V grade, and 1 mM EDTA) for 2 hrs at 37° C. Finally, gels were hybridized overnight with a γ-[32P]-ATP-labeled (TTAGGG)₃ oligonucleotide probe, washed three times in 0.1×SSC for 15 min at 37° C., and exposed to a PhosphorImager screen overnight.

### Results

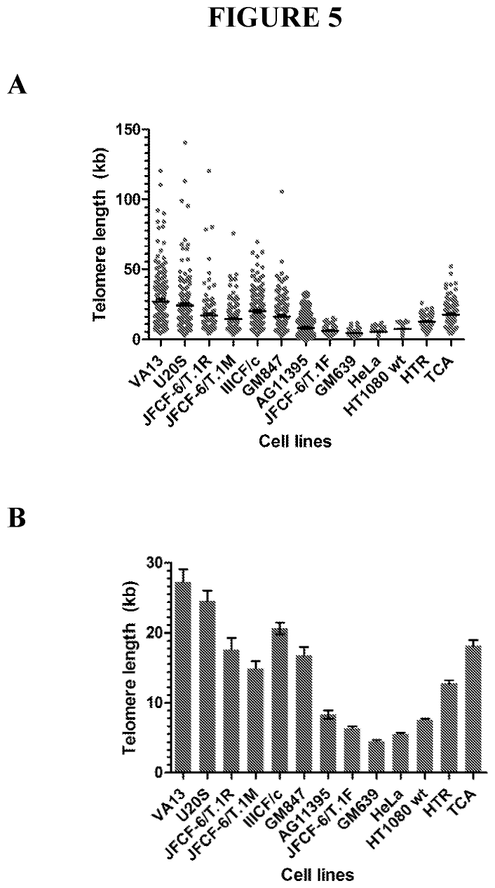

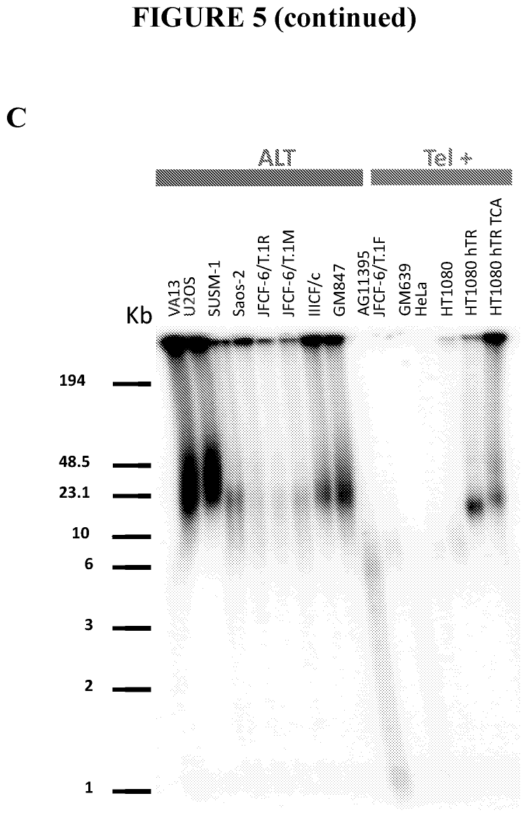

As shown in FIG. 5, telomere length measurements obtained using TFF and TRF analysis on ALT cells and Tel+ cells correlated reasonably well. However, TFF provides significant advantages to TRF, in particular the ability to determine the absolute length of each individual telomere (FIGS. 5A and B). In contrast, TRFs are visualized as a smear, representative of all the individual telomere lengths from every chromosome end in a cell population, i.e. only an average length or range of lengths can be determined (FIG. 5C). Moreover, as TRF length analysis involves using frequently-cutting restriction enzymes to digest the genomic DNA, leaving the telomere fragment (or terminal restriction fragment: TRF) intact, an unknown and variable amount of telomere-adjacent DNA will be included in the TRF measurement, resulting in an overestimation of telomere length. Thus, TFF provides a significant improvement over the current “gold-standard” technique for measuring telomere length.

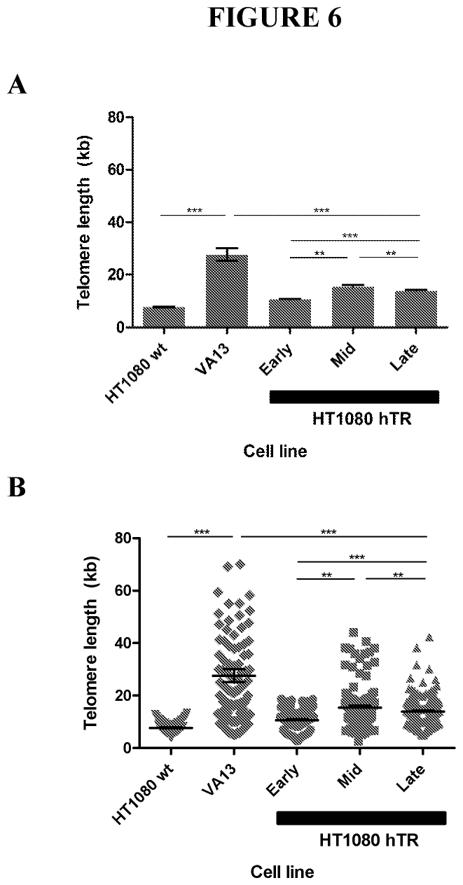

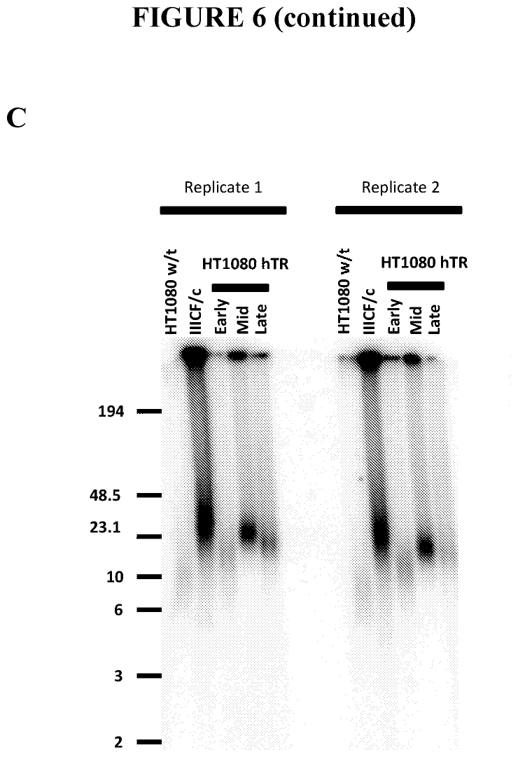

The side-by-side comparison of TFF and TRF length analysis in the HT1080 telomerase-positive cell line also indicated that there was good correlation between telomere length measurements achieved by TFF compared to TRF analysis (FIG. 6). HT1080 wild-type, increasing population doublings of HT1080 hTR cells (early, mid, late), and either the VA13 or the IIICF/c ALT cell line were assayed. The HT1080 hTR cells overexpress the hTR RNA component of telomerase, and therefore elongate their telomeres with progressive population doublings (early to late), until telomere lengths plateau due to telomere shortening by telomere trimming (late). This change in telomere length was observed using both TFF (FIGS. 6A and B) and TRF length analysis (FIG. 6C). However, measurement of telomere length using TFF provided more information on the precise range of telomere lengths and the individual telomere lengths.

C. Comparison of TFF and Flow-FISH and qPCR Techniques

A side-by-side comparison of TFF and flow-FISH (the technique used most commonly in the clinical setting) and qPCR was performed. The lengths of telomeres from HeLa, HT-1080, U2-OS and IIIF/c cells were analysed by each of the techniques.

### Methods

TFF was performed essentially as described above. Briefly, cells were isolated by trypsinization, and 7.5×10⁵ cells were embedded in agarose plugs (1.2% low-melting temperature agarose at 1:1 ratio to cells (45 μL)) to minimise DNA breaks, and subjected to proteinase K digestion (plugs were incubated overnight in a solution of 0.5 M EDTA pH 8.0, 10% (v/v) Sarcosyl/0.5 M EDTA and 20 mg/mL Proteinase K before being washed four times with a 10 mM Tris-HCl and 1 mM EDTA solution (pH 8.0) in rotation followed by incubation in 2-(N-Morpholino)ethanesulfonic acid (MES) pH 5.5 solution at 68° C. for 20 minutes, then 42° C. for 10 minutes, and then with agarose overnight at 42° C.). Molecular combing was performed using the Molecular Combing System (Genomic Vision S.A.) with a constant stretch factor of 2 kb/μm using vinyl silane coverslips (20×20 mm; Genomic Vision S.A.), according to the manufacturer's instructions. After combing, coverslips were dried for 4 hrs at 60° C. Coverslips were fixed with 4% formaldehyde, washed with water and then with PBS, before being serially dehydrated with ethanol (70%, 90% and 100%). Coverslips were labelled in a humidity chamber box with 0.3 ng/mL telomere PNA probe TelC [TAMRA-OO-(CCCTAA)₃] in a PNA hybridization solution (70% formamide, 0.25% NEN blocking reagent, 10 mM Tris pH 7.5, 5% MgCl₂ buffer [82 mM Na₂HPO₄, 9 mM citric acid, 25 mM MgCl₂]). Coverslips were incubated at room temperature overnight and counterstained by first washing with a sequence of three saline-sodium citrate-based detergent: 50% formamide/2×SSC (NaCl and Na₃C₆H₅O₇), followed by 2×SSC and then 0.1% Tween/2×SSC. Coverslips were counterstained with YOYO-1 solution in a humidity chamber box for 20 min at 37° C., before washing with a solution of 1% Tween and 2×SSC, followed by water and air drying. Telomere fibres were detected on a Zeiss Axio Imager microscope with ApoTome module and analyzed with Zen software (Zeiss) or using the Genomic Vision FiberStudio platform. For each cell line, three coverslips were scored and a fourth was partially scored. The number of telomeres assessed for each cell line was as follows: HeLa—463; HT-1080—457; U2-OS—458; IIICF/c—465.

Flow-FISH was performed at Sydney Children's Hospitals Network Department of Haematology Telomere Length Testing Facility. Cells were hybridised with a fluorescein isothiocyanate (FITC)-conjugated (CCCTAA)₃ peptide nucleic acid probe (Panagene, Daejeon, Korea). FITC-labelled fluorescent molecules of equivalent soluble fluorochrome (MESF) calibration beads were used to calibrate the flow cytometer and to establish a fluorochrome-based standard curve. At the beginning and end of each experiment, fluorescence signals from calibration beads suspended in PBS/0.1% BSA were acquired. These beads have known numbers of fluorochrome molecules on their surfaces, varying from 3,000 to 50,000 MESF units. The resulting calibration curve was then used to convert telomere fluorescence data to MESF units, allowing comparison of results among experiments. CCRF-CEM cells were included as a reference sample in each flow-FISH experiment. Experiments were accepted when MESF units showed a variation coefficient (CV) of 10% (±10%). All samples were analysed on a FACS CANTO II (BD Biosciences, San Jose, Calif.) instrument and data displayed and analyzed with BD FACSDiva software (BD Biosciences). Flow-FISH was performed on three replicates of each cell line (HeLa, HT-1080, U2-OS and IIICF/c cell lines). Results are expressed as Molecules of Equivalent Soluble Fluorochrome (MESH), with CCRF-CEM cells used as the control cell line to generate the fluorescence intensity standard for assessing MESH.

qPCR was performed according to Cawthon et al., 2002 Nucleic Acid Res. 2002; 30(10):e47. Briefly, qPCR was conducted in triplicate using Rotor-Gene SYBR Green Master Mix (Qiagen, Chadstone, Australia). Telomere primers were: Tel forward [5′-CGGTTT(GTTTGG)₅GTT-3′] (SEQ ID NO:18) and Tel reverse [5′-GGCTTG(CCTTAC)₅CCT-3′] (SEQ ID NO:19). Single copy gene primers were 36B4 forward [5′-CAGCAAGTGGGAAGGTGTAATCC-3′] (SEQ ID NO:20) and 36B4 reverse [5′-CCCATTCTATCATCAACGGGTACAA-3′] (SEQ ID NO:21) (Invitrogen, Mt Waverley, Australia). Amplification was completed in a Rotor-Gene Q (Qiagen) qPCR cycler. A standard curve was performed for each run using U-2 OS cell line DNA. The telomere content for each sample was determined using the telomere to single copy gene ratio (T/S ratio) by calculating the ΔCt (Ctₜₑₗₒₘₑᵣₑ/Ct_{single copy gene}). The T/S ratio of each sample was normalized to the mean T/S ratio of a reference sample (U-2 OS), which was included in all runs. The experiment was accepted if the reference sample T/S ratio ranged within the 95% variation interval, and if the standard curve equation had at least R²>0.95 and efficiency=1 (±10%). qPCR was performed on three replicates of each cell line in triplicate (HeLa, HT-1080, U2-OS and IIICF/c cell lines).

### Results

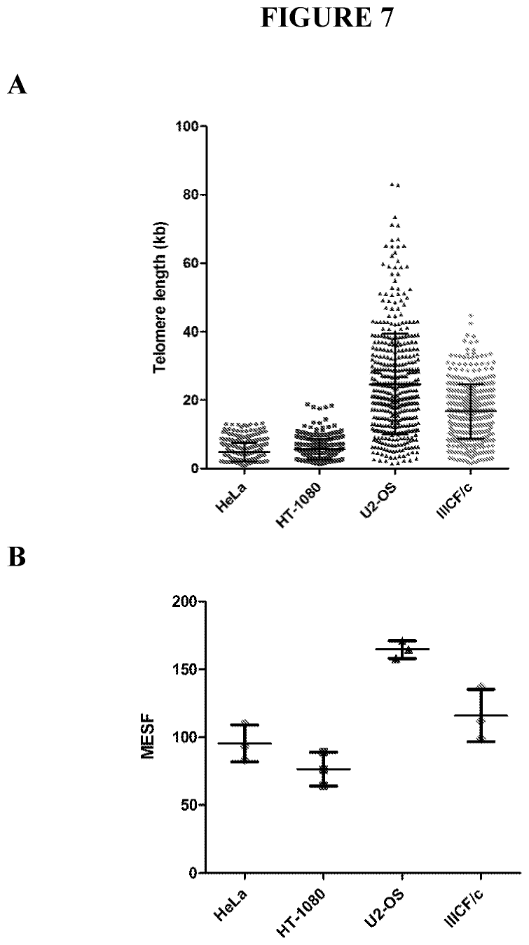

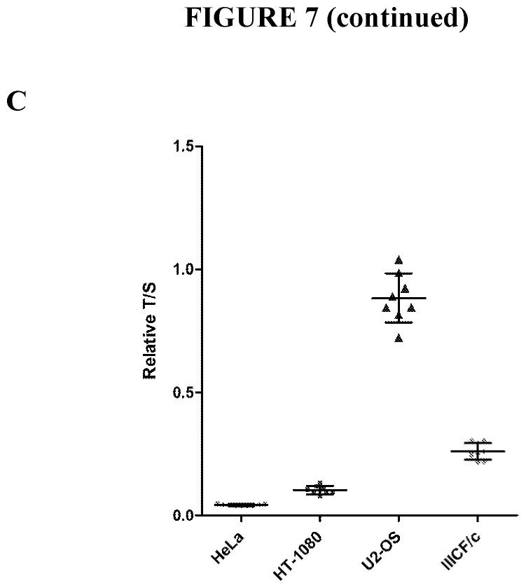

As shown in FIG. 7, each technique demonstrated the heterogeneity in telomere length between cell types, although the average telomere lengths of the cell types relative to one another varied slightly depending on the technique used to measure telomere length. Importantly, TFF provided significant advantages to both flow-FISH and qPCR in that it facilitated absolute measurement of individual telomere lengths, allowing identification and quantification of the extreme short and long telomeres in the samples. In contrast, qPCR and flow-FISH provided only relative average lengths in the cell/cell sample.

### D. Use of TFF to Measure Telomere Length Following Exposure to Inhibitors

TFF was used to measure telomere length in cells exposed to one or both of two inhibitors: aphidicolin and an mTOR inhibitor.

### Methods

Cells (IMR90 primary human foetal lung fibroblasts) were plated and one day later were exposed to 0.1 μM aphidicolin, 0.2 μM of INK128 (an mTOR inhibitor), aphidicolin+INK128, or DMSO alone (untreated). Cells were harvested a further 7 days later and telomere length was assessed by TFF, essentially as described above.

### Results

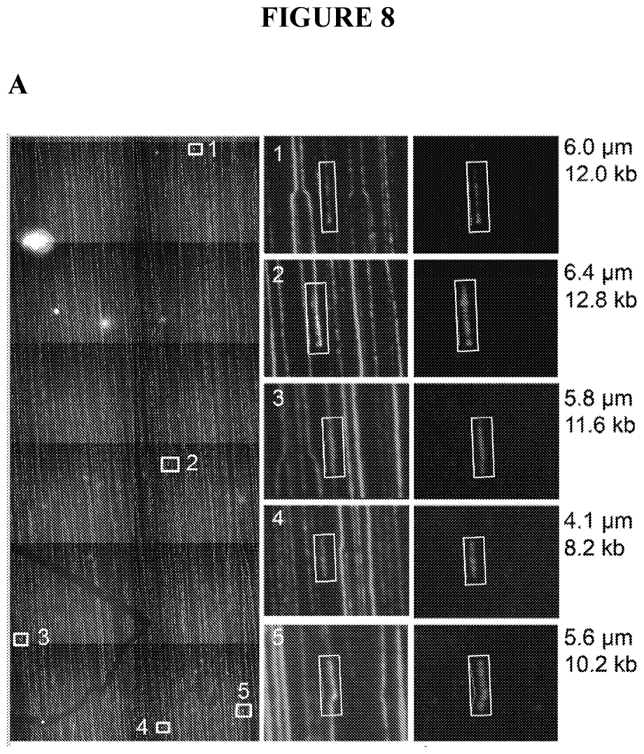

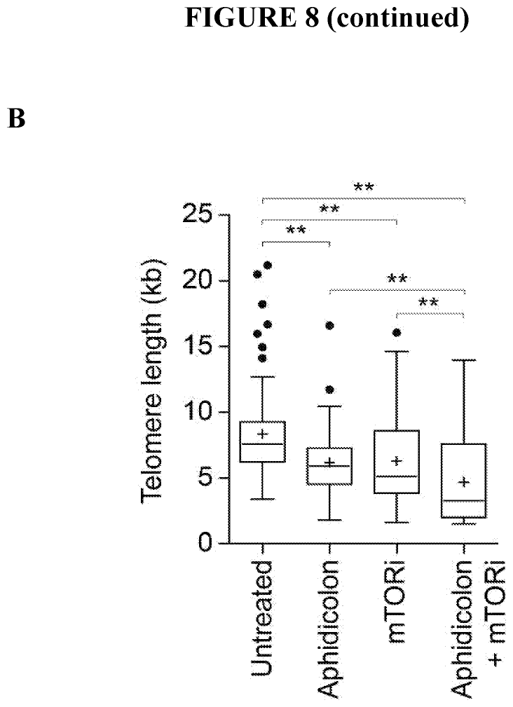

As shown in FIG. 8, the telomere length in untreated cells was significantly greater than cells treated with either or both of aphidicolin and INK128, and the use of both inhibitors resulted in a significant reduction in telomere length compared to the use of one of the inhibitors. As can also be seen from FIG. 8, there was a large degree of heterogeneity in the samples, as also seen in the previous studies.

### Example 2

### Analysis of Telomere Extension Events Using Single Molecule Analysis of Telomeres (SMAT)

The TFF technique described above was adapted to analyse telomere extension events. The resulting method, Single Molecule Analysis of Telomeres (SMAT), can be used to measure the frequency of telomere extension events, the length of telomere extension products, and the length of telomeres prior to extension, and can be applied to cells in culture.

SMAT involves pulsing cells with a nucleotide analogue, such as the thymidine analogue CldU, which is readily incorporated into newly synthesized DNA in place of thymidine. CldU incorporation at telomeres is then visualized on stretched DNA fibres via immunofluorescence and telomere-FISH. Telomere fibres with no CldU incorporation can be scored as non-replicating, telomere fibres with CldU incorporation at the telomere and in a telomere-adjacent region can be categorized as replicating, while telomeric fibres with CldU incorporation strictly at one end of the telomere with no overlap into telomere-adjacent regions can be scored as telomere synthesis events (FIG. 9). The presence of a telomere extension event, and the length of the extension products, can be determined.

The utility of SMAT was assessed in a study designed to directly visualize telomere synthesis events following stable expression of SLX4 (a molecular scaffolding protein) and BLM (a RecQ helicase) in ALT cells.

### Methods

### Generation of Stable Cell Lines

Myc-DDK—tagged SLX4, Myc-DDK—tagged BLM, and pCMV6 empty vectors were obtained from Origene Technologies.

U-2 OS and IIICF/c ALT cells were seeded at 2×10⁵ per 6-well plate and reverse transfected with 1 μg DNA plasmid using FuGENE-6 reagent (Promega) according to the manufacturer's instructions. G418 (400 μg/ml) was added after 24 hrs for selection. Cells were continuously cultured in G418 to ensure overexpression. Protein overexpression was confirmed via western blot analysis and immunofluorescence. SLX4 and BLM overexpressing cell lines are denoted by SLX4+ and BLM+ respectively.

### Single Molecule Analysis of Telomeres (SMAT)

Cells were labelled with 100 μM CldU for 5 hrs. Cells were then isolated by trypsinization, embedded in agarose plugs to minimise DNA breaks, and subject to proteinase K digestion essentially as described above. Molecular combing was performed using the Molecular Combing System (Genomic Vision S.A.) with a constant stretch factor of 2 kb/μm using vinyl silane coverslips (20×20 mm; Genomic Vision S.A.), according to the manufacturer's instructions. After combing, coverslips were dried for 4 hrs at 60° C. Quality and integrity of combed DNA fibres were checked using the YoYo-1 counterstain as described above. Coverslips were denatured for 25 min in alkali-denaturing buffer (0.2M NaOH, 0.1% β-mercaptoethanol in 70% ethanol) and fixed by addition of 0.5% glutaraldehyde for 5 min. Telomeric DNA was visualized by hybridization with a TAMRA-OO-KKK(TTAGGG)₃ PNA probe (Panagene). Halogenated nucleotides were detected with a rat anti-CldU monoclonal antibody (Accurate) and Alexa Fluor 488-conjugated goat anti-rat antibody (Molecular Probes). Telomere fibres were detected on a Zeiss Axio Imager microscope with ApoTome module and analyzed with Zen software (Zeiss).

### Results

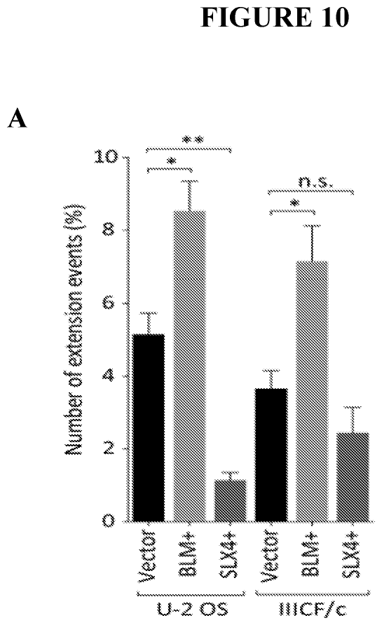

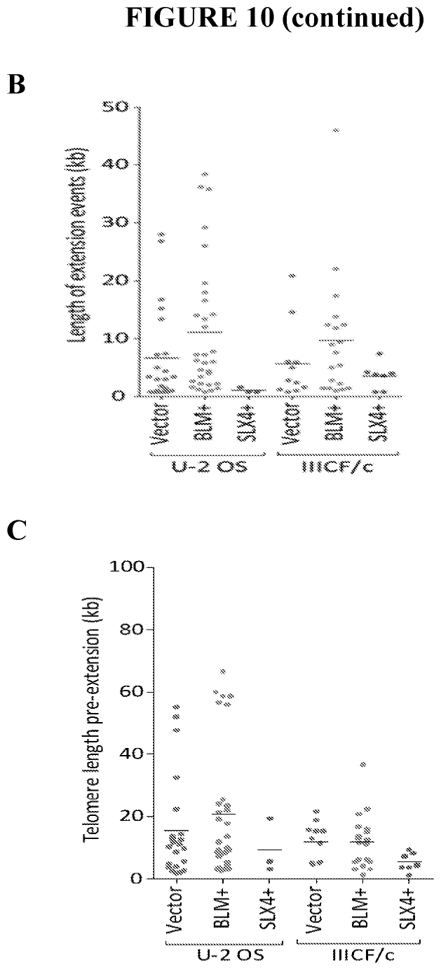

SMAT was effectively employed to detect the number of extension events in cells overexpressing BML or SLX4, the length of any telomere extension products, and the length of the telomere pre-extension. Using SMAT, it was observed that overexpression of BLM increased the number of telomere extension events in both U-2 OS and IIICF/c ALT cell lines, whereas overexpression of SLX4 reduced the frequency of these events (FIG. 10A). Similarly, the absolute length of telomere extension products, calculated based on the constant rate of stretching applied to the DNA fibres (2 kb/μm), was increased in BLM overexpressing ALT cell (ranging from 1 to 45 kb), while telomere extension products were shorter in SLX4 overexpressing ALT cells (FIG. 10B).

### Example 3

### Processing Systems for Methods for Measuring Teleomere Length

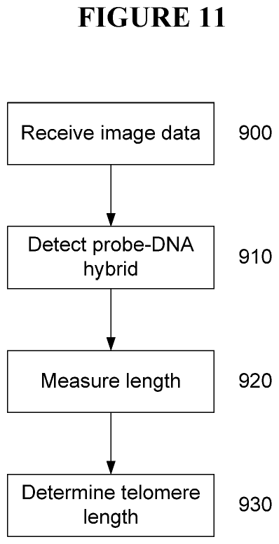

An example of a method for measuring the length of the telomere will now be described with reference to FIG. 11.

In this example, it is assumed that the process is performed at least in part using an electronic processing device forming part of a processing system, which is in turn connected to one or more other computer systems via a network architecture, as will be described in more detail below.

In this example, the one or more processing device receive image data from an imaging device step 900. The image data is typically indicative of one or more images of at least part of a surface of a support, with the surface having stretched genomic DNA hybridized to a telomere-specific probe attached thereto. The image data may be collected in any appropriate manner and may include a single image or multiple images which can be tiled together to form a single composite image. The image data may be received directly from the measuring device, or alternatively may be retrieved from an intervening store, such as a memory database or the like.

At step 910 a probe-DNA hybrid is detected in the one or more images. This typically involves having the one or more processing devices perform image analysis techniques in order to identify the probe-DNA hybrid within the image and an example of these will be described in more detail below.

The one or more processing devices measure a length of the probe-DNA hybrid in the one or more images at step 920. The measurement can be performed in any appropriate manner and may simply involve performing a length measurement in the image, but alternatively could involve aligning the probe-DNA hybrid with a pixel direction and then counting a number of pixels or measuring the fluorescence intensity of the pixels in the probe-DNA hybrid.

The measured length is then used to determine a telomere length indicator indicative of the telomere length at step 930, for example basing this on the measured length, or scaling the measured length as required, depending on the nature of the image and the imaging device. The indicator can be stored and/or displayed to an operator as needed.

Accordingly, it would be appreciated that this provides a mechanism in order to allow one or more processing devices to automatically process the image data in order to detect the telomere length. This avoids the need to perform the manual processes outlined above.

A number of further features will now be described.

In one example, the method includes detecting a probe-DNA hybrid in accordance with pixel parameters of image pixels of the image. The pixel parameters can include any one or more of pixel color, pixel intensity, pixel brightness, pixel hue or pixel saturation. Most typically the probe-DNA hybrid is detected through fluorescence and accordingly is identified utilizing pixel intensity. It will be appreciated however that this is not essential and any one or more of the abovementioned pixel parameters could be used. It will be appreciated that these techniques allow the probe-DNA hybrid to be automatically identified in the image. To assist with this process, the method typically includes determining the length of the probe-DNA hybrid utilizing region growing and/or edge detection techniques. For example, a part of the probe-DNA hybrid can be located, with region growing then being used to identify the full extent of the probe-DNA hybrid in the images.

In one particular example this is achieved by detecting candidate features in the image using the pixel parameters, and then detecting a probe-DNA hybrid at least partially in accordance with the candidate features. This is typically achieved by determining an extent of the candidate feature and then detecting the probe in accordance with the extent, for example by comparing this to one or more thresholds. The comparison can take into account a width and/or length, or alternatively a width to length ratio with this being used to ascertain whether the candidate feature is likely to be a probe-DNA hybrid, or is another feature or image artefact. In this regard, it will be appreciated that there will be well understood expected ranges for the probe-DNA hybrid and if extent of the candidate feature falls outside the expected ranges, it will be excluded from further analysis. In one particular example, this takes into account feature size, location and orientation in order to exclude candidate features with the probe-DNA hybrid being detected from the remaining features, although any suitable assessment can be used.

In order to improve the effectiveness of the detection process, the image may undergo image enhancement to generate an enhanced image. Such enhancement can include contrast enhancement, hue enhancement, intensity enhancement, brightness enhancement, colour enhancement, saturation enhancement or the like. The probe-DNA hybrid is then detected using the enhanced image. This is performed as this can increase the accuracy of the probe-DNA hybrid detection.

As previously mentioned in one example a plurality of images are captured in which case the method can include forming a composite image from the plurality of images and detecting a probe-DNA hybrid using the composite image. The need for use of composite images will depend on factors such as the field of view of the imaging device and the size of substrate surface to which the probes are attached.

Whilst the measured length can be used directly as an indication of telomere length, in another example, a length of a plurality of probe-DNA hybrids measured with these means statistically analyzed to determine a telomere length and/or a telomere length distribution. This can be performed, for example to account for the fact that some telomeres may break during combing and to allow variations in telomere length to be analyzed.

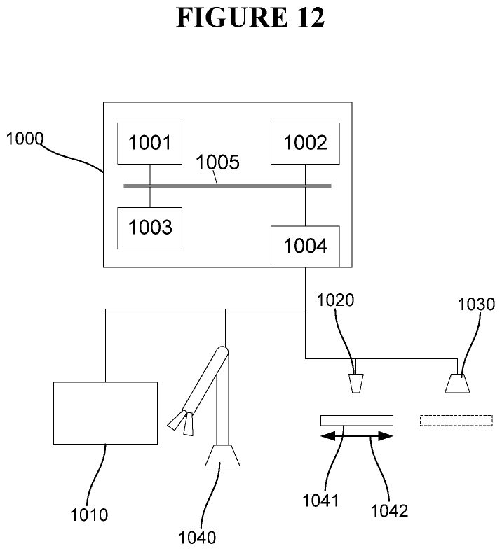

An example of a system for use in automatically measuring telomere length will now be described with reference to FIG. 12.

In this example, the system includes a processing system 1000, a combing machine 1010, an applicator 1020, an imaging device 1030 and a transport system optionally including one or more transport elements such as a manipulator arm 1040 and a moveable stage 1041, moveable as shown in multiple directions as shown for example by arrows 1042.

In this example, the processing system 1000 includes an electronic processing device, such as at least one microprocessor 1001, a memory 1002, an optional input/output device 1003, such as a keyboard and/or display, and an external interface 1004, interconnected via a bus 1005 as shown. In this example the external interface 1004 can be utilized for connecting the processing system 1000 to peripheral devices, such communications networks 1000, storage devices such as databases, as well as the combing machine, transport system and imaging device. Although a single external interface 1004 is shown, this is for the purpose of example only, and in practice multiple interfaces using various methods (eg. Ethernet, serial, USB, wireless or the like) may be provided.

In use, the microprocessor 1001 executes instructions in the form of applications software stored in the memory 1002 to perform required processes, such as controlling the other equipment, and processing image data to determine a telomere length. Thus, actions performed by a processing system 1000 are performed by the processor 1001 in accordance with instructions stored as applications software in the memory 1002 and/or input commands received via the I/O device 1003, or commands received from other processing or computer systems. The applications software may include one or more software modules, and may be executed in a suitable execution environment, such as an operating system environment, or the like.

Accordingly, it will be appreciated that the processing systems 1000 may be formed from any suitable processing system, such as a suitably programmed computer system, PC, web server, network server, or the like. In one particular example, the processing systems 1000 are standard processing system such as a 32-bit or 64-bit Intel Architecture based processing system, which executes software applications stored on non-volatile (e.g., hard disk) storage, although this is not essential. However, it will also be understood that the processing system could be or could include any electronic processing device such as a microprocessor, microchip processor, logic gate configuration, firmware optionally associated with implementing logic such as an FPGA (Field Programmable Gate Array), or any other electronic device, system or arrangement.

The combing machine 1010 is typically known as a molecular combing machine, such as a Genomic Vision Systems Molecular Combing Platform. The applicator 1020 can be any computer controlled system for delivering the probe, whilst the imaging device 1030 is typically a camera, CCD sensor or the like provided as part of a microscope. The transport system can include a slide loading manipulator art 1040, and moveable stage, as known in the art.

In use, the processing system 1000 can operate the combing machine in order to allow combing to be performed. The manipulator arm 1040 is then used to load the substrate onto the stage 1041 allowing this to be transported to the applicator 1020, which applies the telomere specific probe to the surface. The stage 1041 can then be moved into alignment with the imaging device 1030, allowing imaging of the probe-DNA hybrids to be performed.

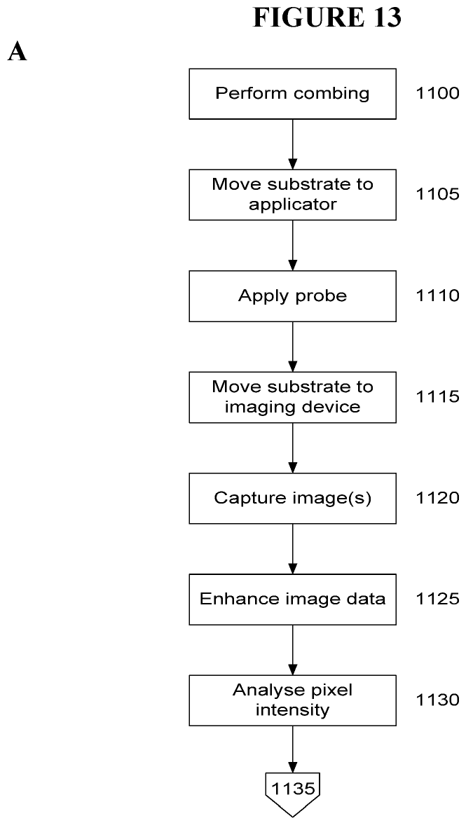

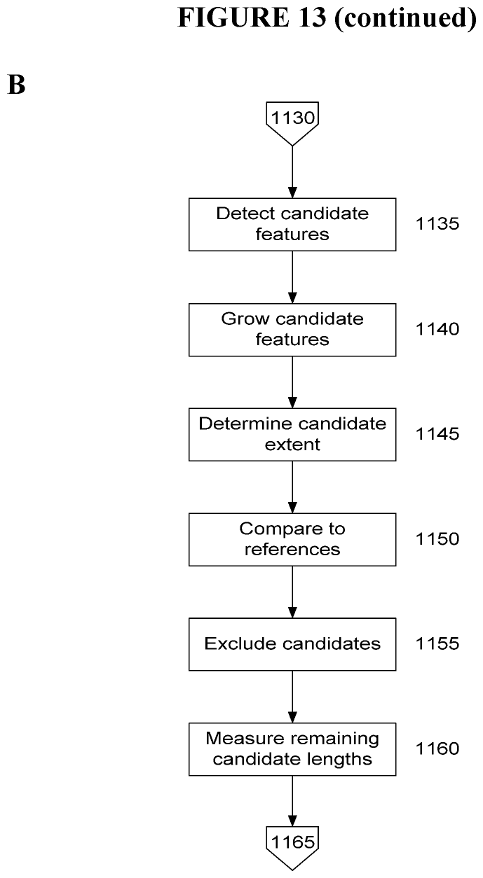

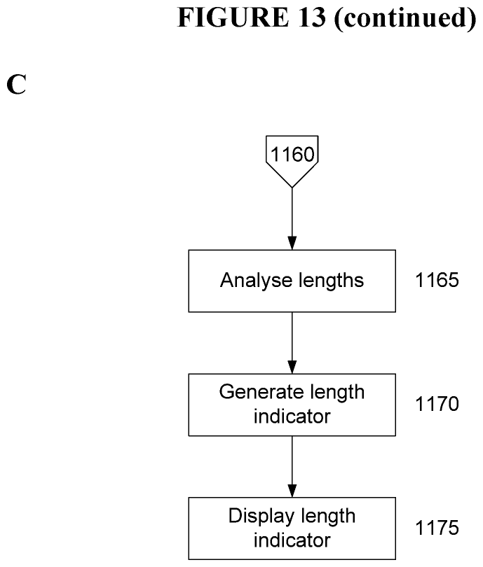

An example process for performing length detection will now be described in further detail with reference to FIG. 13.

In this example, at step 1100 combing is performed utilizing the combing machine 1010. Following this, the manipulator arm 1040 is used to mount the substrate on to the stage 1041 which is moved into alignment with the applicator 1020. The probe is applied at step 1110 before the substrate is moved into alignment with the imaging device step 1115, allowing one more images to be captured at step 1120, with the image data being transferred to the processing system 1000 for analysis.

At step 1125, the processing system 1000 enhances the image by performing contrast or other suitable forms of enhancement, before pixel intensities are analyzed at step 1130. In particular, the pixel intensities are examined to identify bright regions which are then labelled as candidate features at step 1135. Candidate features are grown in order to detect candidate feature boundaries at step 1140, with the boundaries being used to determine extents of the candidate features at step 1145. This will typically involve determining a length and width of the candidate features although other information such as a shape and orientation may also be derived.

The candidate extents are then compared to reference thresholds allowing candidates to be excluded at step 1155. The lengths of any remaining candidates are measured at step 1160 with the length being statistically analyzed at step 1165. This process will typically involve excluding any outlier values, typically corresponding to broken telomeres, and then calculating a mean and standard deviation of the remaining telomeres. This is used to generate a length indicator at step 1170, which can then be displayed and/or stored at step 1175.

The disclosure of every patent, patent application, and publication cited herein is hereby incorporated herein by reference in its entirety.

The citation of any reference herein should not be construed as an admission that such reference is available as “Prior Art” to the instant application.

The reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavour to which this specification relates.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1. A method for measuring the length of a telomere, comprising:
stretching genomic DNA on to a surface of a support at a uniform stretching rate;
hybridizing a telomere-specific probe to the DNA to obtain a probe-DNA hybrid; and
detecting the probe-DNA hybrid to thereby determine the length of a telomere.

2. The method of claim 1, wherein the length of the telomere is determined by visualizing and measuring the absolute length of the probe-DNA hybrid.

3. The method of claim 1, wherein the length of the telomere is determined by measuring the intensity of a detectable signal on the telomere-specific probe within the probe-DNA hybrid.

4. A method for detecting extension of a telomere, comprising:
contacting a cell with a nucleotide analogue under conditions sufficient for DNA synthesis and incorporation of the nucleotide analogue into the genomic DNA of the cell;
isolating the genomic DNA from the cell;
stretching the genomic DNA on to a surface of a support at a uniform stretching rate;
hybridizing a telomere-specific probe to the DNA to obtain a probe-DNA hybrid; and
detecting the probe-DNA hybrid and the nucleotide analogue to thereby determine whether telomere extension has occurred, wherein the presence of a probe-DNA hybrid comprising the nucleotide analogue is indicative of the extension of a telomere.

5. The method of claim 4, further comprising measuring the length of the extension, the length of the telomere with the extension and/or the length of the telomere excluding the extension.

6. The method of claim 5, wherein measuring the length of the extension, the length of the telomere with the extension and/or the length of the telomere excluding the extension is performed by visualizing and measuring the absolute length of the probe-DNA hybrid.

7. The method of claim 5, wherein measuring the length of the extension, the length of the telomere with the extension and/or the length of the telomere excluding the extension is performed by measuring the intensity of a detectable signal on the telomere-specific probe within the probe-DNA hybrid.

8. The method of any one of claims 4 to 7, further comprising contacting the cell with an agent that inhibits or promotes telomere extension.

9. The method of any one of claims 1 to 8, wherein the uniform stretching rate is between 0.1 kb/μm and 10 kb/μm, between 0.5 kb/μm and 5 kb/μm, or between 1 kb/μm and 3 kb/μm.

10. The method of any one of claims 1 to 9, wherein the uniform stretching rate is between 1.5 kb/μm and 2.5 kb/μm.

11. The method of any one of claims 1 to 10, wherein the uniform stretching rate is 1.6, 1.7, 1.8, 1.9, 2.0, 2.1, 2.2, 2.3 or 2.4 kb/μm.

12. The method of any one of claims 1 to 11, wherein the genomic DNA is stretched on to the surface by molecular combing.

13. The method of any one of claims 1 to 12, wherein the genomic DNA is stretched on to the surface by a molecular combing machine.

14. The method of any one of claims 1 to 13, wherein the telomere-specific probe hybridizes to one or more telomere repeat units.

15. The method of claim 14, wherein the genomic DNA is human genomic DNA and the telomere repeat unit comprises the sequence TTAGGG.

16. The method of any one of claims 1 to 15, wherein the telomere-specific probe is a nucleic acid probe comprising the sequence CCCTAA or TTAGGG.

17. The method of any one of claims 1 to 16, wherein the telomere-specific probe is a peptide nucleic acid (PNA) probe.

18. The method of any one of claims 1 to 17, wherein the telomere-specific probe comprises a visually-detectable label.

19. The method of claim 18, wherein the visually-detectable label is a fluorescent label.

20. The method of any one of claims 4 to 19, wherein the nucleotide analogue comprises a visually-detectable label.

21. The method of claim 20, wherein the visually-detectable label is applied to the analogue after incorporation of the analogue into the DNA.

22. The method of claim 20, wherein the nucleotide analogue comprises the visually-detectable label prior to incorporation of the analogue into the DNA.

23. The method of any one of claims 20 to 22, wherein the visually-detectable label is a fluorescent label.

24. The method of any one of claim 4 to 21 or 23, wherein the nucleotide analogue is a thymidine analogue selected from among chlorodeoxyuridine (CldU), bromoeoxyuridine (BrdU), iododeoxyuridine (IdU) and ethynyldeoxyuridine (EdU).

25. The method of any one of claim 4 to 20, 22 or 23, wherein the nucleotide analogue is a fluorescent analogue selected from 2-aminopurine (2AP), pyrrolo-C (PyC), 1,3-diaza-2-oxophenothiazine (tC), 1,3-diaza-2-oxophenoxazine (tC°) and 7-nitro-1,3-diaza-2-oxophenothiazine (tCₙᵢₜᵣₒ).

26. The method of any one of claims 1 to 25, further comprising staining the genomic DNA.

27. The method of claim 26, wherein the genomic DNA is stained with a fluorescent dye.

28. The method of claim 27, wherein the fluorescent dye is selected from among YOYO-1, TOTO-1, POPO-1, BOBO-1 and JOJO-1.

29. The method of any one of claims 1 to 3, wherein the genomic DNA is obtained from frozen, fresh, or fixed cells or tissue.

30. The method of any one of claims 1 to 3, wherein the method first comprises a step of extracting genomic DNA from cells or tissue.

31. The method of claim 30, wherein the cells or tissue are frozen, fixed or fresh.

32. The method of any one of claims 1 to 31, wherein the support is a glass support.

33. The method of claim 32, wherein the glass support is silanized.

34. The method of claim 32 or 33, wherein the glass support is coated with vinyl silane.

35. The method of any one of claims 32 to 34, wherein the glass support is a microscope slide or coverslip.

36. The method of any one of claims 1 to 35, wherein at least a portion of the method is automated.

37. A method for measuring the length of a telomere, the method including, in one or more processing devices:
receiving image data captured by an imaging device, the image data being indicative of one or more images of at least part of a surface of a support, the surface having stretched genomic DNA hybridized to a telomere-specific probe attached thereto;
detecting a probe-DNA hybrid in the one or more images;
measuring a length of the probe-DNA hybrid in the one or more images; and,
using the measured length to determine a telomere length indicator indicative of the telomere length.

38. A method according to claim 37, wherein the method includes detecting a probe-DNA hybrid in accordance with pixel parameters of image pixels of the image, the pixel parameters including at least one of:
a pixel colour;
a pixel intensity;
a pixel brightness;
a pixel hue; and,
a pixel saturation.

39. A method according to claim 37 or claim 38, wherein the method includes determining the length of the probe-DNA hybrid by at least one of:
region growing; and,
edge detection.

40. A method according to any one of the claims 37 to 39, wherein the method includes:
detecting candidate features in the image using the pixel parameters; and,
detecting a probe-DNA hybrid at least partially in accordance with the candidate features.

41. A method according to claim 40, wherein the method includes:
determining an extent of a candidate feature; and,
detecting a probe-DNA hybrid in accordance with the determined extent.

42. A method according to claim 41, wherein the method includes:
comparing the extent to one or more thresholds; and,
detecting a probe-DNA hybrid in accordance with results of the comparison.

43. A method according to claim 41 or claim 42, wherein the method includes determining the extent by at least one of:
region growing; and,
edge detection.

44. A method according to any one of the claims 41 to 43, wherein the method includes:
selectively excluding candidate features in accordance with at least one of:
candidate feature size;
candidate feature location; and,
candidate feature orientation; and,
detecting a probe-DNA hybrid from the remaining candidate features.

45. A method according to any one of the claims 37 to 44, wherein the method includes:
performing enhancement to generate an enhanced image using at least one of:
contrast enhancement;
hue enhancement;
intensity enhancement;
brightness enhancement;
colour enhancement; and,
saturation enhancement; and,
detecting a probe-DNA hybrid using the enhanced image.

46. A method according to any one of the claims 37 to 45, wherein the image data includes a plurality of images and wherein the method includes:
forming a composite image from the plurality of images; and,
detecting a probe-DNA hybrid using the composite image.

47. A method according to any one of the claims 37 to 46, wherein the method includes:
measuring a length of plurality of probe-DNA hybrids; and,
statistically analyzing the measured lengths to determine at least one of:
a telomere length; and,
a telomere length distribution.

48. A system for measuring the length of a telomere, the system including, one or more processing devices that:
receive image data from an imaging device, the image data being indicative of one or more images of at least part of a surface of a support, the surface having stretched genomic DNA hybridized to a telomere-specific probe attached thereto;
detect a probe-DNA hybrid in the one or more images;
measure a length of the probe-DNA hybrid in the one or more images; and,
use the measured length to determine a telomere length indicator indicative of the telomere length.

49. A system according to claim 48, wherein the one or more processing devices:
generate an indicator indicative of at least one of:
a telomere length; and,
a telomere length distribution; and,
at least one of display and store the indicator.

50. A system according to claim 48 or claim 49, wherein the one or more processing devices control the imaging device to capture the one or more images.

51. A system according to any one of the claims 48 to 50, wherein the support is mounted on a stage, and the one or more processing devices control actuators to thereby relatively move the stage and imaging device to thereby capture the one or more images.

52. A system according to any one of the claims 48 to 51, wherein the system includes:
a molecular combing machine that stretches the genomic DNA on to the surface;
an applicator that applies the telomere-specific probe to the surface; and,
at least one transport mechanism for transporting the substrate from the molecular combing to the applicator and from the applicator to the stage.

53. A system according to claim 52, wherein the one or more processing devices control at least one of:
the molecular combing machine;
the applicator; and,
the at least one transport mechanism.

54. A computer program product including computer executable code, which when executed by a suitably programmed processing system causes the processing system to:
receive image data from an imaging device, the image data being indicative of one or more images of at least part of a surface of a support, the surface having stretched genomic DNA hybridized to a telomere-specific probe attached thereto;
detect a probe-DNA hybrid in the one or more images;
measure a length of the probe-DNA hybrid in the one or more images; and,
use the measured length to determine a telomere length indicator indicative of the telomere length.


SEQUENCE LISTING (17 sequences)

SEQ ID NO: 1 - DNA, 6 bp, Homo sapiens
ttaggg

SEQ ID NO: 2 - DNA, 6 bp, Homo sapiens
ccctaa

SEQ ID NO: 3 - DNA, 7 bp, Arabidopsis thaliana
tttaggg

SEQ ID NO: 4 - DNA, 10 bp, Cestrum elegans
ttttttaggg

SEQ ID NO: 5 - DNA, 12 bp, Allium ursinum
ctcggttatggg

SEQ ID NO: 6 - DNA, 8 bp, Chlamydomonas reinhardtii
ttttaggg

SEQ ID NO: 7 - DNA, 5 bp, Bombyx mori
ttagg

SEQ ID NO: 8 - DNA, 12 bp, Artificial sequence
  Probe 1
ccctaaccctaa

SEQ ID NO: 9 - DNA, 18 bp, Artificial sequence
  Probe 2
ccctaaccctaaccctaa

SEQ ID NO: 10 - DNA, 12 bp, Artificial sequence
  Probe 3
ttagggttaggg

SEQ ID NO: 11 - DNA, 18 bp, Artificial sequence
  Probe 4
ttagggttagggttaggg

SEQ ID NO: 12 - DNA, 6 bp, Homo sapiens
tcaggg

SEQ ID NO: 13 - DNA, 6 bp, Homo sapiens
tgaggg

SEQ ID NO: 14 - DNA, 6 bp, Homo sapiens
taaggg

SEQ ID NO: 15 - DNA, 6 bp, Homo sapiens
ccctga

SEQ ID NO: 16 - DNA, 6 bp, Homo sapiens
ccctca

SEQ ID NO: 17 - DNA, 6 bp, Homo sapiens
ccctta